(19) United States
(12) Patent Application Publication
(10) Pub. No.: US 2004/0030339 A1
(43) Pub. Date: Feb. 12, 2004

(54) Dual locking plate and associated method

(76) Inventors: Michael A. Wack, Warsaw, IN (US); Pamela C. Guzman, Fort Wayne, IN (US); Dennis A. Stoller, Fort Wayne, IN (US); Christopher K. Bremer, Warsaw, IN (US); Mark A. Fenton, North Manchester, IN (US); Lawrence B. Bone, Buffalo, NY (US); Roy W. Sanders, Tampa, FL (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/633,469

(22) Filed: Aug. 1, 2003

Related U.S. Application Data
(63) Continuation-in-part of application No. 10/100,387, filed on Mar. 18, 2002.
(60) Provisional application No. 60/285,462, filed on Apr. 20, 2001.

(51) Int. Cl. A61B 17/56

(52) U.S. Cl. 606/69

(57) ABSTRACT

A fracture repair system for engagement with a bone having a condylar portion and a shaft portion is provided. The system includes a plate. The plate includes a head portion and a body portion. The head portion has an internal wall defining a head hole therethrough and is adapted for cooperation with the condylar portion. The body portion has an internal wall defining a body hole through the wall. The system further includes a bushing having a generally spherical exterior surface adapted for cooperation with the head hole and an opposed interior surface defining a passageway through the bushing. The exterior surface of the bushing and the head hole of the plate are configured to permit polyaxial rotation of the bushing within the head hole. The system also includes a head attachment component including a distal portion sized for clearance passage through the passageway and into the bone and an opposed proximate portion sized to urge the bushing against the internal wall of the plate to form a friction lock between the bushing and the plate in a selected polyaxial position. The head attachment component is positionable in an orientation extending divergingly from the plate. The system also includes a body attachment component having a stem portion for passage through the body hole and into the bone and an opposed cap portion sized to cooperate with the plate.

### CROSS REFERENCE TO RELATED APPLICATIONS

This application is a Continuation-in-Part of U.S. patent application Ser. No. 10/100,387 filed Mar. 18, 2002, entitled POLYAXIAL LOCKING PLATE. U.S. patent application Ser. No. 10/100,387 is a Utility Application based upon U.S. Provisional Patent Application, Serial No. 60/285,462 filed Apr. 20, 2001, entitled POLYAXIAL LOCKING PLATE.

### BACKGROUND OF THE INVENTION

The present invention relates to a bone locking plate, more particularly the present invention relates to a bone locking plate that includes an adjustable attachment component. Most particularly, the present invention relates to a bone locking plate that includes an attachment component whose angle relative to the locking plate may be manipulated during surgery so that an accompanying screw extends into the bone in a desirable orientation.

The skeletal system includes many long bones which extend from the human torso. These long bones include the femur, fibula, tibia, humerus, radius and ulna. These long bones are particularly exposed to trauma from accidents and as such often are fractured during such trauma and may be subject to complex devastating fractures.

Automobile accidents for instance are a common cause of trauma to long bones. In particular the femur and tibia frequently fracture when the area around the knee is subjected to a frontal automobile accident.

Often the distal and/or proximal portions of the long bone, for example, the femur and tibia are fractured into several components and must be re-attached.

Mechanical devices most commonly in the form of pins, plates and screws are commonly used to attach fractured long bones. The plates, pins and screws are typically made of a durable material compatible with the human anatomy, for example titanium, stainless steel or cobalt chrome. The plates are typically positioned longitudinally along the periphery of the long bone and have holes or openings through which screws may be inserted into the long bone transversely. Additionally, intramedullary nails or screws may be utilized to secure fractured components of a long bone, for example, to secure the head of a femur.

Fractures of long bones typically occur in high stress areas, for example, near the condyles or distal or proximal portions of the long bones. Such fractures in the distal or proximal condyle portions of the long bone may result in many individual fragments which must be reconnected. Optimally, the bone plates should be positioned adjacent to the distal or proximal portions of the long bones and permit the securing of these fragments.

More recently bone plates have been provided for long bones which have a profile which conforms to the distal or proximal portion of the long bone. For example such bone plates are available from DePuy ACE in the form of supra condylar plate systems. These plates have a contoured periphery to match the distal portion of a long bone, for example, a femur. These plates, however, include holes or opening through which transverse screws are used to secure the bone plate to the long bone. The openings in the bone plate provide thus for only one general orientation of the screw for attachment of the bone fragments, which is normally or perpendicularly to the bone plate. Thus often the optimum position of a screw may not be utilized as it does not conform to a position nominal or perpendicular to the bone plate.

Often with a fracture of condyles of the distal portion of a long bone the adjacent screws should be positioned and locked in a divergent direction diverging from the bone plate so that the distal condyles may be properly secured by the bone screw. Two dimensional bone plates do not provide for the optimum diverging orientation of the bone screws.

Recently DePuy Acromed, Inc. has developed locking plates, as disclosed in U.S. Pat. No. 5,954,722 to Bono, for use in spinal applications which include a pivotable bushing within the plate which bushing is internally threaded and mates with external threads on bone screws. This type of locking plate permits an orientation of the bone screw in a position other than normally with the bone plate while also permitting locking of the screw.

Proper securement of a bone plate to a bone is dependent on, among other things, the condition of the bone. For example, if the bone is severely fractured, the fasteners are preferably unlocking or not rigidly secured to the plate. By not locking the fastener to the plate, the fastener can be used to pull or draw the fragments of the fractured bone together to assist in blood flow and the healing of the fracture site. Such non-locking fasteners may include, for example, fasteners with cancellous threads to securely contain the fragments. Non-locking fasteners may also include a portion of the stem which is not threaded or be in the form of a lagging screw to assist in the drawing of bone fragments together. Further, the use of a non-locking fastener results in increased flexion on motion between the fasteners and the plate thereby increasing the stress or load on the fracture site. Such increase in fracture load or bracing of the stress adjacent to fracture site results in hypertrophy or the increase in size of the cortical bone due to the physical activity to accommodate the higher stress. Such a reaction to the increased stress at the fracture site is well borne out by Wolff's Law.

Locking fasteners, for example, locking screws, however, provide for a more rigid construction and may provide an alternate construction for a bone plate and may be used in bone of any quality. For example, if the bone of the patient is osteoporotic or has a thin cortical layer or an eggshell cortical layer, the increased stress due to flexion between the fasteners and the bone plate caused by movable or unlocked fasteners, may fracture the cortical bone and not support such a construction. Thus, for osteoporotic bone, the use of fasteners locked to the bone plate is preferred. While x-rays and other analytical tools may be utilized to determine the type of bone of the patient, the actual condition of the bone of the patient may not be fully determined until the fracture sight is exposed. Thus, there is a need to interoperatively provide a plate which may be selectively locked or unlocked with respect to its fasteners.

Occasionally, when a fastener is used to secure a bone plate, the fastener is screwed into osteoporotic or otherwise weak bone and the fastener may become stripped or not properly secured into the bone. The fastener may be removed and a different location or bone site may be necessary to secure the plate with the fastener.

Occasionally, a bone plate will lift up or separate from the bone. This is particularly a problem with the portion of the bone plate opposite the head or condylar portion of the bone plate. As the patient moves, for example, walks, the bone plate flexes and the portion of the bone plate moves toward and away from the bone. This motion may cause the plate to loosen from the bone.

Attempts have been made to implant bone plate percutaneously, or implant the bone plate with a minimal incision in the skin. Problems have occurred in properly and securely moving the bone plate adjacent the bone to percutaneously position it in the proper location.

### SUMMARY OF THE INVENTION

According to the present invention, a fracture repair system is provided that includes a bone plate having separate, spaced apart, locking and non-locking openings. The locking and non-locking openings are used in conjunction with locking and non-locking fasteners. The fracture repair system of the present invention may be utilized for both healthy bone and osteoporotic bone. The surgeon may, during the installation of the bone plate, make a final decision as to the choice of locked and unlocked securement of the plate. The decision may be influenced by an observation as to the condition of the bone. The surgeon can provide, in the alternative, locking and non-locking securement of the plate to the bone.

According to the present invention, the fracture repair system of the present invention provides for a bone plate that may accommodate a first fastener with a first portion for engagement with the bone plate and a second portion for engagement with the bone. If the portion of the bone used for engagement with the screw becomes stripped or does not properly secure the fastener to the bone, the first fastener may be removed and second larger fastener may be utilized. The second larger fastener may include a first portion which is substantially identical to the first portion of the first fastener and is lockable to the bone plate. The second, larger fastener also includes a portion for engagement with the bone which is larger that the corresponding bone securing portion of the first fastener. Thus, the second, larger fastener may be utilized if the bone adjacent the first fastener becomes stripped. The second, larger fastener may include cancellous threads in the bone engaging portion, while the first fastener may include cortical threads in the bone engaging portion.

According to the present invention, a fracture repair system for engagement with a bone may include a plate with a polyaxial bushing. The polyaxial bushing cooperates with a screw having a tapered cap to cooperate with the axial bushing. The polyaxial bushing and mating fastener may be utilized to provide for diverging orientation of the bone screws to accommodate fracture of the condyles.

According to the present invention a fracture repair system may include a plate having a distal hole for cooperation with a fastener for rigid attachment of the plate to the bone distally and opposed to the condylar portion of the bone plate. The rigid attachment at the distal hole serves to assist in preventing motion of the plate toward and away from the bone if the plate is spaced from or lifted off of the plate opposed to the condylar portion of the plate. The spacing or lifting off of the plate from the bone may be the result of either difficulty securing the screw into the cortical bone or due the non-conformity of the plate to the bone shaft.

According to an embodiment of the present invention, a fracture repair system for engagement with a bone having a condylar portion and a shaft portion is provided. The system includes a plate. The plate includes a head portion and a body portion. The head portion has an internal wall defining a head hole therethrough and is adapted for cooperation with the condylar portion. The body portion has an internal wall defining a body hole through the wall. The system further includes a bushing having a generally spherical exterior surface adapted for cooperation with the head hole and an opposed interior surface defining a passageway through the bushing. The exterior surface of the bushing and the head hole of the plate are configured to permit polyaxial rotation of the bushing within the head hole. The system also includes a head attachment component including a distal portion sized for clearance passage through the passageway and into the bone and an opposed proximate portion sized to urge the bushing against the internal wall of the plate to form a friction lock between the bushing and the plate in a selected polyaxial position. The head attachment component is positionable in an orientation extending divergingly from the plate. The system also includes a body attachment component having a stem portion for passage through the body hole and into the bone and an opposed cap portion sized to cooperate with the plate.

According to another embodiment of the present invention a fracture repair system for engagement with a bone having a condylar portion and a shaft portion is provided. The system includes a plate including a head portion and a body portion. The body portion has an internal wall defining a first body hole and a spaced apart second body hole through the plate. The system further includes a rigid body attachment component including a stem portion for passage through the first body hole and into the bone and an opposed cap portion adapted to rigidly cooperate with the plate The system also includes a movable body attachment component including a stem portion for passage through the second body hole and into the bone and an opposed cap portion adapted to movably cooperate with the plate.

Still further, in accordance with the present invention a fracture repair system for engagement with a bone having a condylar portion and a shaft portion is provided. The system includes a plate including a head portion and a body portion. The body portion has an internal wall defining a body hole the plate. The system further includes a first rigid body attachment component including a stem portion for clearance passage through the body hole and into the bone and an opposed cap portion adapted to rigidly cooperate with said plate. The system also includes a second rigid body attachment component including a stem portion for threadable engagment with the body hole and into the bone and an opposed cap portion adapted to rigidly cooperate with said plate.

Further, in accordance with the present invention a fracture repair system for engagement with a bone is provided. The fracture repair system includes a plate having a portion having an internal wall defining a first body hole and a spaced apart second body hole through the plate. The system further includes a rigid body attachment component including a stem portion for passage through the body hole and into the bone and an opposed cap portion adapted to rigidly cooperate with said plate. The system also includes a movable body attachment component including a stem portion for passage through the body hole and into the bone and an opposed cap portion adapted to movably cooperate with said plate.

Also, in accordance with the present invention a joint fracture system for use with joint having adjoining first and second long bones is provided. The system includes a first plate for cooperation with the first long bone. The first plate has a first plate head portion and a first plate body portion. The first plate body portion has an internal wall defining a first plate first body hole and a spaced apart first plate second body hole through the plate. The system also includes a first plate rigid body attachment component including a stem portion for passage through the first plate body hole and into the bone and an opposed cap portion adapted to rigidly cooperate with said first plate. The system also includes a first plate movable body attachment component including a stem portion for passage through the first plate body hole and into the bone and an opposed cap portion adapted to movably cooperate with said first plate. The system also includes a second plate for cooperation with the second long bone. The second plate includes a second plate head portion and a second plate body portion. The second plate body portion has an internal wall defining a second plate first body hole and a spaced apart second plate second body hole through the plate. The system also includes a second plate rigid body attachment component including a stem portion for passage through the second plate first body hole and into the bone and an opposed cap portion adapted to rigidly cooperate with said second plate. The system also includes a second plate movable body attachment component including a stem portion for passage through the second plate body hole and into the bone and an opposed cap portion adapted to movably cooperate with said second plate.

Still further, in accordance with the present invention a method for repairing a bone fracture on a bone having a condylar portion and a shaft portion is provided. The method includes the steps of providing a locking plate apparatus including moveable body attachment component, a fixed body attachment component and a plate having a head portion and a body portion and at least two plate holes through the body portion, the first plate hole for rigid attachment to the plate and the second plate hole for moveable attachment to the plate. The method further includes the steps of determining which a locked and a non-locked plate construction should be used selecting the fixed body attachment component if a locked plate should be used and selecting the moveable body attachment component if a non-locked plate should be used inserting the fixed body attachment component into the first plate hole if the locked plate should be used and inserting the moveable body attachment component into the second plate hole if the non-locked plate should be used, and securing the fixed body attachment component if the locked plate should be used and securing the moveable body attachment component if the non-locked plate should be used.

Also, in accordance with the present invention, a fracture repair system for engagement with a bone having a condylar portion and a shaft portion is provided. The system includes a plate having a head portion and a body portion. The head portion has an internal wall defining a head hole therethrough and adapted for cooperation with the condylar portion. The body portion has an internal wall defining a body hole therethrough. The system also includes an attachment component including a distal portion sized for passage through at least one of the head hole and the body hole and into the bone and an opposed proximate portion sized to rigidly secure to the internal wall of one of the head hole and the body hole. The distal portion is generally cylindrical and has a smooth periphery.

The technical advantages of the present invention include the ability to interoperatively select between rigid and movable securement of the plates. For example, according to one aspect of the present invention, the fracture repair system of the present invention includes a bone plate that includes threaded holes and spaced apart clearance holes on the body of the plate. The threaded holes cooperate with fasteners having a threaded cap for rigid securement of the fastener to the plate. The fracture repair system further includes movable fasteners that include caps which are movably secured at the clearance holes on the body of the plate. Thus, the present invention provides for an interoperatively or in situ selection of rigid or movable securement of the plate.

Another technical advantage of the present invention is that the surgeon may interoperatively or in situ in the patient replace a fastener that has become stripped in the fracture repair system with a larger screw and maintain the rigid securement of the plate. For example, according to one aspect of the present invention, the fracture repair system includes a bone plate that has a threaded hole on the body of the bone plate. The fracture repair system further includes a first fastener which has threads on the cap portion of the fastener as well as small cortical threads on the stem portion of the fastener. If the bone mating with the cortical threads on the stem portion of the fastener becomes stripped, the first fastener may be removed from the bone plate and a second larger fastener, which has a threaded portion on the cap portion of the larger fastener with threads identical to that of the smaller fastener as well as cancellous larger threads on the stem portion of the second larger fastener. Thus, the present invention provides for interoperative use of a larger screw with rigid securement of the plate if the bone mating with the first installed smaller screw is stripped.

Yet another advantage of the present invention is that the bone fragments separated by trauma may be reconnected. For example, according to one aspect of the present invention, the fracture repair system of the present invention may include a plate having holes into which lag screws may be fitted. The fracture repair system further may include a lag screw or a screw having a portion of the stem void of threads. If the first bone fragment is connected to the head of the fastener and the second bone fragment is connected to the threaded portion of the lag screw as the lag screw is rotated, the bone fragments may be connected or drawn together. Thus, the present invention provides for the connection of separated bone fragments and resulting improvement of blood flow and healing.

The technical advantages of the present invention further include the ability to position a screw in a divergent direction diverging from the bone plate so that distal condyles and fragments thereof may be properly secured by the bone screw. For example, according one aspect of the present invention, a fracture repair system is provided which includes a bone plate with a cooperating bushing having a spherical periphery. The bushing therefore may be spherically rotated with respect to the bone plate and the bushing may receive a bone screw or fastener which may be fixedly secured at any position by tightening the bone screw to the plate utilizing the split bushing. Thus, the present invention provides for lockably securing a bone plate in diverging directions.

Additional objects, features, and advantages of the invention will become apparent to those skilled in the art upon consideration of the following detailed description of the preferred embodiment exemplifying the best mode of carrying out the invention as presently perceived.

### BRIEF DESCRIPTION OF THE DRAWINGS

FIG. 1 is a plan lateral view of a fracture repair system in accordance with the present invention with a femur plate coupled to a femur and a tibia plate coupled to a tibia;

FIG. 2 is a perspective lateral view of a fracture repair system in accordance with the present invention with a femur plate coupled to a femur and a tibia plate coupled to a tibia;

FIG. 3 is a perspective view of a femur plate in accordance with the present invention;

FIG. 4 is a plan view of a the femur plate of FIG. 3;

FIG. 5 is an enlarged plan view of a the femur plate of FIG. 3;

FIG. 6 is a cross section view of the femur plate of FIG. 5 taken along lines 6-6;

FIG. 6A is a cross section view of the femur plate of FIG. 5 taken along lines 6A-6A;

FIG. 7 is a perspective view of a tibia plate in accordance with the present invention;

FIG. 8 is a plan view of a the tibia plate of FIG. 7;

FIG. 9 is a cross section view of the tibia plate of FIG. 8 taken along lines 9-9;

FIG. 10 is a plan view of a cannulated, cancellous bone screw for attachment to cancellous bone of a long bone for use with the fracture repair system of FIG. 1;

FIG. 11 is a partial cross sectional view of the tibia plate of FIG. 8 taken along lines 11-11 showing a portion of the tibia plate coupled to the tibia with the tibia plate in cross section and showing various bone screws positioned in the tibia;

FIG. 12 is a partial cross sectional view of the femur plate of FIG. 4 taken along lines 12-12 showing a portion of the femur plate coupled to the femur with the femur plate in cross section and showing two of the bone screws of FIG. 14 positioned in divergent positions in the condyles of the femur in accordance with the present invention;

FIG. 12A is a partial cross sectional view of the femur plate of FIG. 4 taken along lines 12A-12A showing a portion of the femur plate coupled to the femur with the femur plate in cross section and showing the bone screw of FIG. 10 positioned in the condyles of the femur;

FIG. 13 is a plan view of a cortical bone screw for attachment to both cortical bone surfaces of a long bone and for engagement with the bone plate for use with the femur plate of FIG. 4;

FIG. 14 is a plan view of a bone screw for engagement with the bone plate installed in a bone plate according to the present invention with a portion of the bone plate and a bushing providing polyaxial rotation shown in cross section;

FIG. 15 is a top view of a bushing for providing polyaxial rotation of the bone screw according to the present invention;

FIG. 16 is a plan view shown in cross section of the bushing of FIG. 15;

FIG. 17 is a plan view of a drill guide instrument installed on a bone plate for use with the fracture repair system of FIGS. 1-16;

FIG. 18 is a plan view of a cancellous bone screw for attachment to cancellous bone of a long bone for use with the fracture repair system of FIG. 1;

FIG. 19 is a plan lateral view of a fracture repair system in accordance with another embodiment of the present invention with a femur plate coupled to a femur and a tibia plate coupled to a tibia with the plates having fully locking and non-locking portions as well as fasteners including the locking fastener of FIG. 23 and the non-locking fastener of FIG. 13;

FIG. 20 is a perspective lateral view of the femur and tibia plate of the fracture repair system of FIG. 19;

FIG. 21 is a plan view of a the femur plate of FIG. 19, as well as, fasteners including the locking cortical screw of FIG. 23, the locking cancellous cortical screw of FIG. 24, the partially threaded lag screw of FIG. 25, the fully threaded cancellous screw of FIG. 18, the non-locking cortical screw of FIG. 13, the polyaxial locking cancellous screw of FIG. 14, the polyaxial locking cancellous screw of FIG. 26 and the cannulated cancellous screw of FIG. 12A;

FIG. 21A is a cross section view of the femur plate of FIG. 21 taken along lines 21A-21A in the direction of the arrows;

FIG. 21B is a cross section view of the femur plate of FIG. 21 taken along lines 21B-21B in the direction of the arrows;

FIG. 21C is a cross section view of the femur plate of FIG. 21 taken along lines 21C-24C in the direction of the arrows;

FIG. 22 is a plan view of the tibial plate of FIG. 19, as well as, fasteners including the locking cortical screw of FIG. 23, the non-locking cortical screw of FIG. 13, and the polyaxial locking cancellous screw of FIG. 14;

FIG. 22A is a cross section view of tibial femur plate of FIG. 22 taken along lines 22A-252 in the direction of the arrows; and

FIG. 23 is a plan view of a cortical bone screw for attachment to cortical bone of a long bone and with proximal locking threads for use with the fracture repair system of FIG. 19;

FIG. 24 is a plan view of a cancellous bone screw for attachment to cancellous bone of a long bone and with larger threads for use to replace the screw of FIG. 23 including proximal locking threads compatible with the proximal locking threads of the screw of FIG. 16 for use with the fracture repair system of FIG. 21;

FIG. 25 is a plan view of a partially threaded cancellous bone screw for attachment to cancellous bone of a long bone to connect bone fragments by lagging with the fracture repair system of FIG. 19;

FIG. 26 is a plan view of a bone screw smaller than the screw of FIG. 14 for engagement with the bone plate of FIG. 19 installed in a bone plate according to the present invention with a portion of the bone plate and a bushing providing polyaxial rotation shown in cross section;

FIG. 27 is a plan view of a K-wire for use with the bone plates of FIGS. 21 and 22;

FIG. 28 is a plan view of a fracture repair system in accordance with another embodiment of the present invention with a femur plate of FIG. 19. for coupling to a femur having fully locking portions and including the locking cortical screw of FIG. 23 and the larger locking cortical screw of FIG. 24; and

FIG. 29 is a flow chart of a method of performing trauma surgery using the fracture repair system in accordance with another embodiment of the present invention.

### DETAILED DESCRIPTION

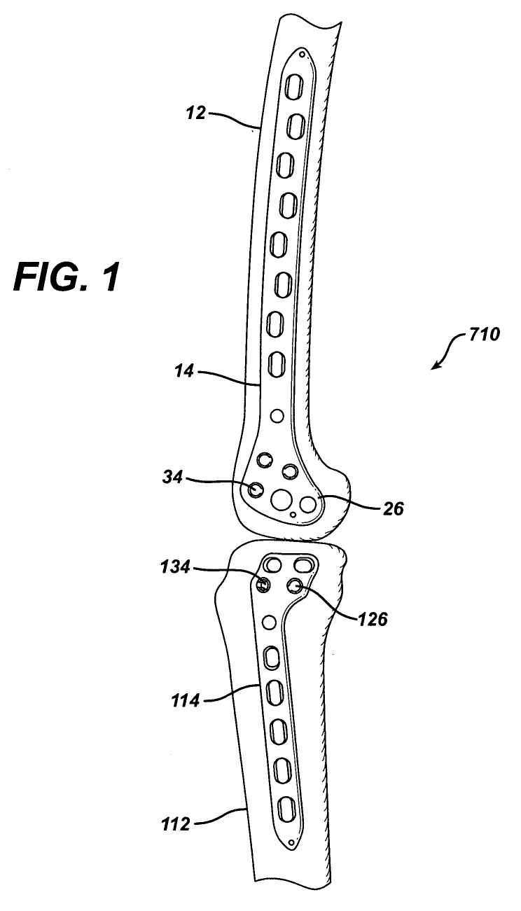

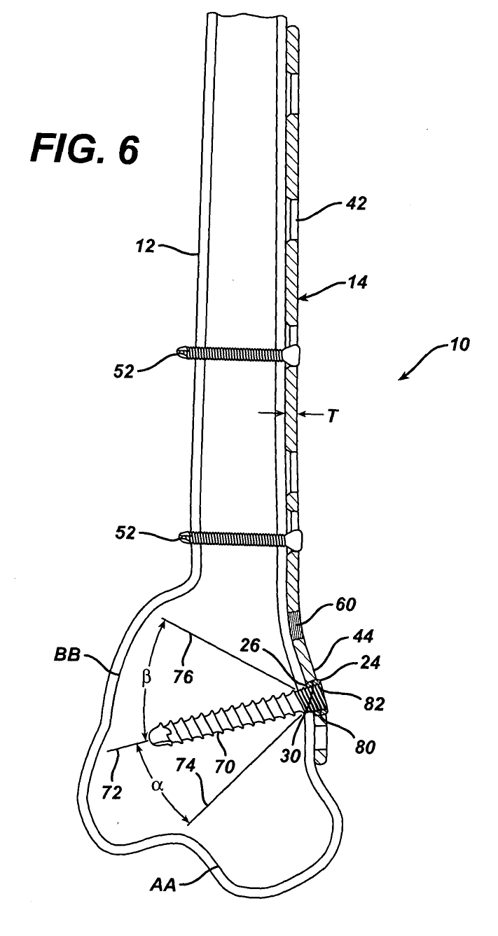

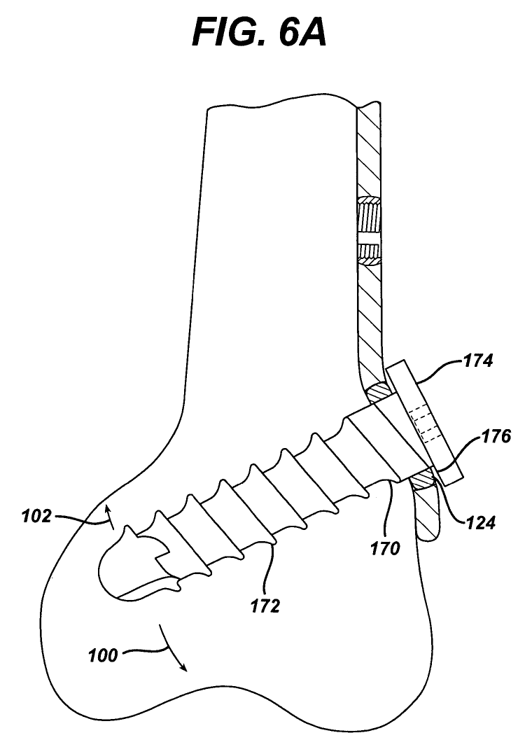

According to the present invention and referring now to FIG.6, a fracture repair system 10 is shown for engagement with a long bone 12. The long bone 12 may be any long bone, for example, a femur, tibia, fibula, humerus, radius or ulna, but as shown in FIG. 1 the long bone is a femur. The fracture repair system 10 includes a plate 14.

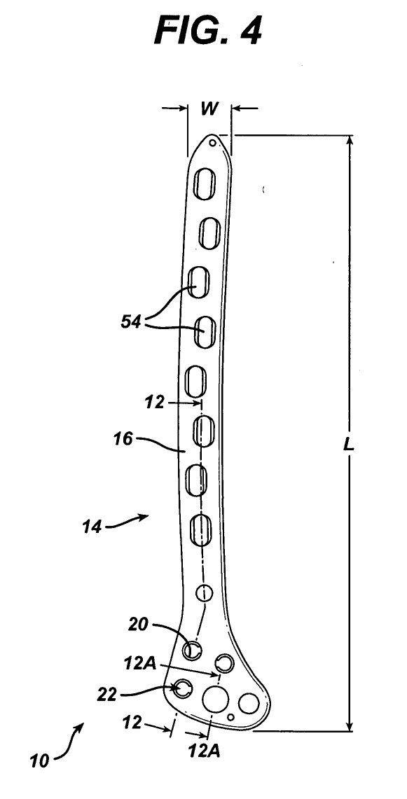

Referring now to FIG. 4 the fracture repair system 14 is shown in greater detail. The plate 14 may be made of any suitable durable material and may, for example, be made of a metal, for example, a metal compatible with the human anatomy, for example, cobalt chrome, stainless steel or titanium. The plate 14 includes a body portion 16 and an interior wall 20. The interior wall 20 defines a plate hole 22 through the body portion 16.

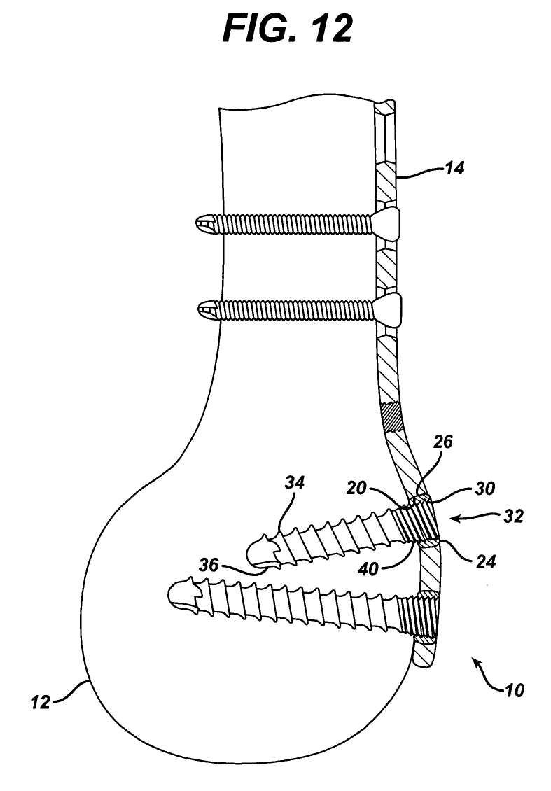

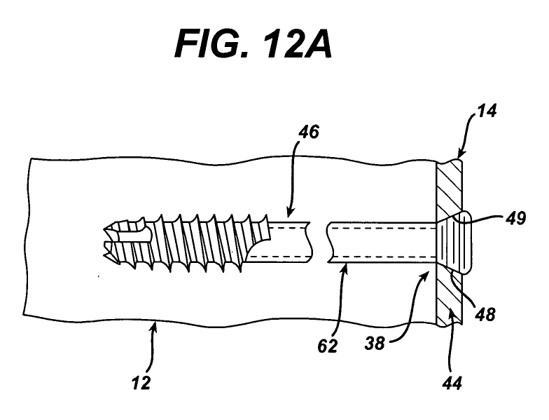

Referring now to FIG. 12 the fracture repair system 10 is shown in greater detail. In addition to the plate 14 the fracture repair system 10 includes one or more bushings 24. The bushing 24 includes a radial exterior surface 26 and opposite radial interior surface 30. The opposite radial interior surface 30 defines a passageway 32 through the bushing 24. The exterior surface 26 of the bushing 24 and the interior wall 20 of the plate 14 are configured to permit the polyaxial rotation of the bushing 24 within the plate hole 22. (see FIG.12) Such polyaxial rotation may be permitted by providing an arcuate or spherical surface on the interior wall 20 of the plate 14 and a mating arcuate or spherical surface on the radial exterior surface 26 of the bushing 24.

The fracture repair system 10 further includes the attachment component 34 includes a distal portion 36 sized for current passage through the passageway 32 and into the long bone 12. The attachment component 34 further includes an opposite proximal portion 40 sized to press the bushing 24 against the internal wall 20 of the plate 14 to form a friction lock between the bushing 24 and the plate 14 in a selected polyaxial position. The attachment component 34 is positionable in an orientation extending divergently from the center of the plate.

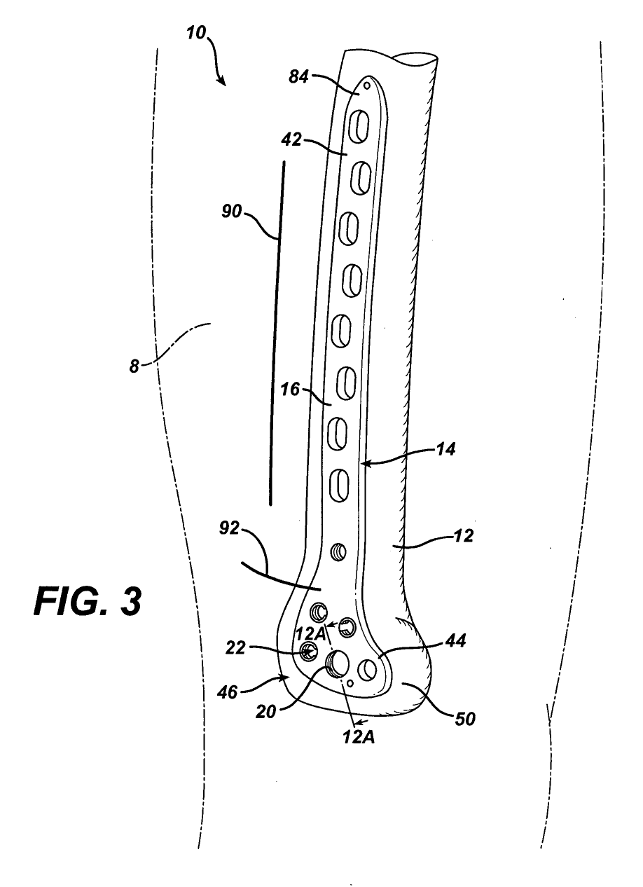

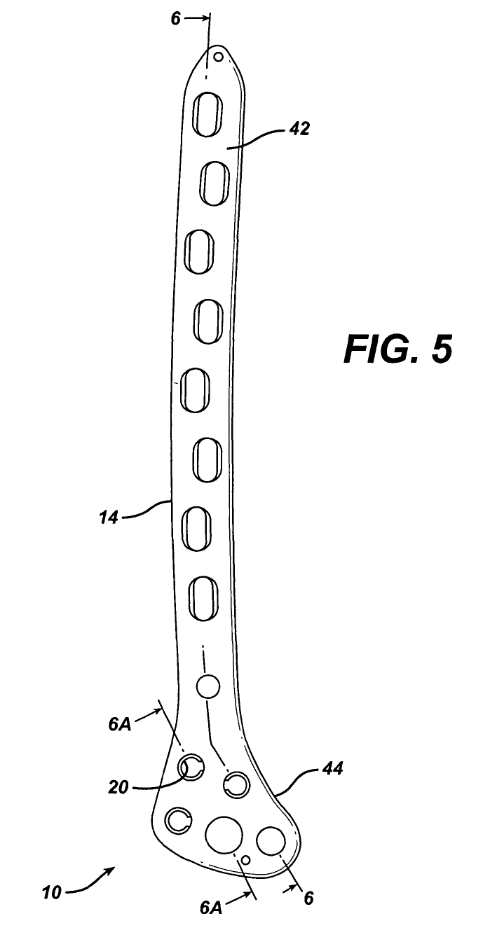

Referring now to FIGS. 3 through 6 the fracture repair system 10 is shown as femur plate assembly 10. Preferably and as shown in FIGS. 3-6, the body portion 16 of the femur plate 14 preferably includes a proximal portion 42 and distal portion 44. To provide for optimal support of the femur, the femur plate 14 has a shape generally conforming to the outer periphery of the femur 12. The proximal portion 42 of the bone plate 14 is generally flat or planer in conforming to the general flat or planer nature of the proximal shaft portion of the femur. The femur plate 14 may have a bow to accommodate the natural anterior/posterior bow of the femur 12. The distal portion 44 of the femur plate 14 has a shape generally conforming to the condylar portion 46 of the femur 12. Since the condylar portion 46 of the femur 12 is arcuate or curved the distal portion 44 of the femur plate 14 is preferably curved to mate with condyles 50 of the condyle portion 46 of the femur 12.

While the particular size and shape and dimensions of the femur plate 14 may vary widely depending upon the size of the femur on which it is installed, for an adult human femur, the plate 14 may have, for example as shown in FIG. 4, an overall length L of about 6 to 14 inches and a width W of about 1¼ to ¾ of an inch and a thickness T of, for example as shown in FIG. 6, about ⅛-¼ inch. Since the human anatomy is generally symmetrical, the femur plate 14 is either a right hand or left hand femur plate and the right hand and left-hand femur plates are different, but generally symmetrical with each other.

While the fracture repair system of the present invention includes one or more bushings which cooperates with an attachment component such that the attachment component may be positionable in an orientation diverging from the center of the plate, it should be appreciated that the fracture system or plate may include a plurality of attachment components. Further these attachment components may be of different styles or types.

Referring now to FIG. 6, the femur plate 14 is shown with three different types of attachment components. Solid, fully-threaded, cortical screws 52 are positioned in elongated openings 54 shown in FIG. 4 in the proximal portion 42 of the femur plate 14. The fully-threaded cortical screws 52 may, as shown in FIG. 6, be self-tapping and cut threads while they are being screwed through the plate into the bone during surgery. The cortical screws 52 are supported primarily by the cortical bone to which they have been secured. While the proximal portion 42 of the bone plate 14 may be secured by a solitary cortical screw 52 preferably and, as shown in FIG. 6, the proximal portion 42 of the femur plate 14 is supported by a series of several spaced apart fully threaded cortical bone screws.

To provide ample support for the proximal portion 42 of the plate 14 and to provide for a standard commercially available femur plate 14, the femur plate 14 preferably includes a uniformly spaced apart pattern of elongated openings 54 shown in FIG. 4. The surgeon may choose any of a number of the elongated openings 54 shown in FIG. 4 in which to drill and screw the cortical screws. Depending on the position of the fractures as few as two or three cortical screws may be sufficient to support the femur plate 14.

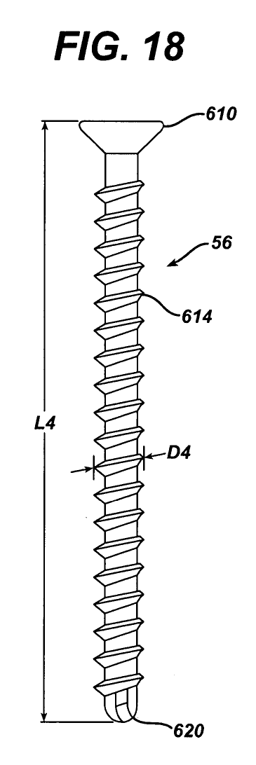

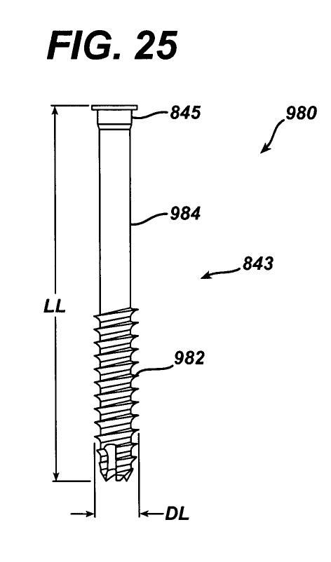

Continuing to refer to FIG. 6, cancellous screws 56 (see FIG. 18) or screw 980 (see FIG. 25) may also be placed in the elongated openings 54 and used to secure the proximal portion 42 of the femur plate 14.

The screw 56 or 980 unlike cancellous screw 70 (see FIG. 6) does not include threads on the head of screw 56 or 980. The lack of screw threads on the head of screw 56 or 980 allows the head to spin on the bushing 24 without locking, thereby achieving a lagging effect. The cancellous screws 56 or 980 may be any suitable size and may, for example, be 2 to 6 millimeters solid, cancellous, partially or fully threaded cancellous screws. The cancellous screws 56 or 980, preferably have a length less than the thickness of the femur so that they may not protrude from the opposite surface of the femur.

Distal portion 44 of the femur plate 14 is designed to follow the general contours of the lateral distal femur while the proximal portion 42 incorporates the natural bow of the femur.

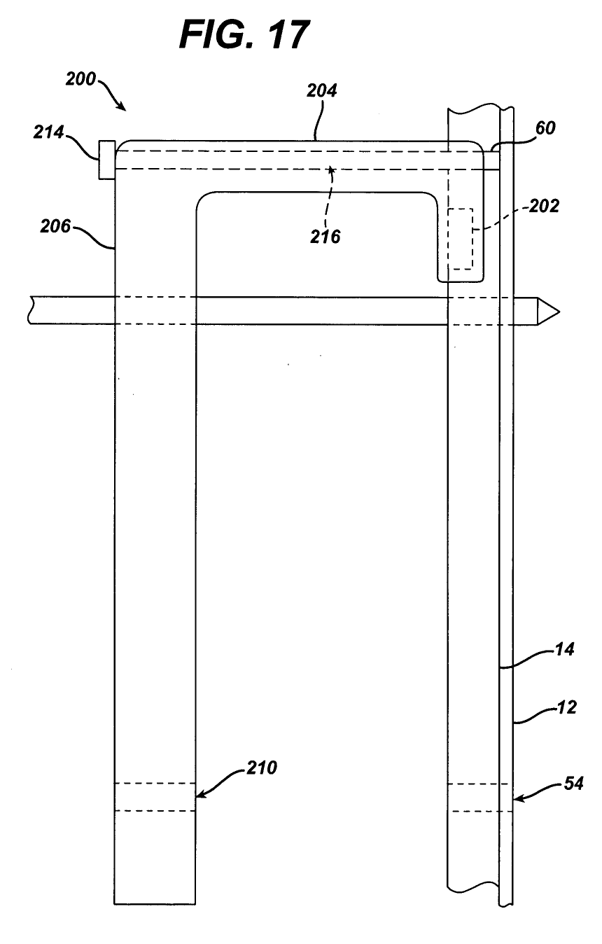

The femur plate 14 may include one or more tapped openings 60 in the femur plate 14 which may be utilized to secure a drill guide 200 shown in FIG. 17 for aligning a drill and a screw driver for the insertion of the screws 52 and 56 or 980 into the femur. The drill guide 200 will be described in greater detail later.

According to the present invention, the plate 14 includes attachment components which are positionable in an orientation diverging from the center of the plate. The plate 14 thus includes at least one screw 70 which is secured to the plate 14 by means of the bushing 24. The screw 70 may be in the form of a cancellous screw. The cancellous screw is particularly well suited for securing the condylar portion of the distal portion of the femur. The cancellous screw 70 may be partially or fully threaded and may have any suitable length to reach the proper portion of the fractured condylar portion of the distal femur. For example, the cancellous screw may have a length from 20 to 150 millimeters. The cancellous screw may have a suitable diameter to properly secure the fractured portions of the femur. For example, the cancellous screw may have a diameter of 3 to 10 millimeters. The cancellous screw 70 is used to secure the distal portion of the femur plate to the bone.

The cancellous screws may be rotated from the first position 72 shown in solid to position 74 shown rotated an angle α or to a third position 76 rotated in the opposite direction an angle β (see FIG. 6). If the cancellous screw 70 is rotated to the second position 74, the screw 70 will be utilized to secure fragment AA while, if the cancellous screw 70 is rotated to position 76, the cancellous screw 70 may be utilized to secure fragment BB. The tip of the cancellous screws 70 can therefore be rotated in a conical pattern.

The cancellous screw 70 as shown in FIG. 6 may include external threads 80 on the head or proximal portion of the screw 70. Alternatively, the head or proximal portion may have a smooth conical head. The external threads 80 mate with internal threads 82 on the bushing 24. Preferably and as shown in FIG. 6 the external threads 80 are tapered such that as the external threads 80 of the screw 70 are engaged into the bushing 24 the bushing 24 expands, locking the radial external surface 26 of the bushing 24 to the radial interior surface 30 of the plate 14.

By permitting the bushing 24 to rotate within the plate 14 and by permitting the bushing 24, the screw 70 and the plate 14 to all be locked securely in place, the screw may be fixedly positioned in many different orientations, while maintaining all components at minimal stress. As shown in FIG. 6, the feature of having the positionable screw and plate configuration permits either fragment AA or fragment BB to be secured by the screw 70.

Referring now to FIG. 6A, one or any portion of the locations of the plate 14 may include one or more bushings 124 which may alternatively be utilized with a screw having a non-threaded head. For example, as shown in FIG. 6A a cancellous screw 170 is shown similar to screw 70 but not including external threads on the head of the screw. The screw 170 does include cancellous threads 172 for securement to the cancellous bone. The screw 170 includes a head 174 which is secured against face 176 of bushing 124. In this configuration the bushing 124 serves to permit the rotation of the screw 170 in the direction of arrows 100 and 102, thus permitting the orientation of the screw 170. The use of the bushing 124 prevents stress risers on the head 174 or face 176 of the bushing 124.

The plate 14 may be made of any suitable durable material that is biologically compatible with the human anatomy and preferable made of a high strength metal. For example, the plate may be made of stainless steel, cobalt chrome or titanium. Preferably the plate 14 is manufactured from a forged or wrought titanium alloy. One such suitable alloy is ASTM F-620-97 and another suitable alloy is ASTM F-136 ELI.

Referring to FIG. 37, the femoral plate 14 may be secured to the femur 12 during surgery either percutaneously or by conventional open surgery. When the femur plate 14 and screws are implanted in conventional open surgery a longitudinal cut 90 is made through the skin along the thigh 8 laterally where the femur plate is typically installed. A lateral installation of the femur plate provides for the minimal interference with muscle, ligaments and other soft tissue. The longitudinal cut 90 in the thigh 8 through the skin parallel to the femur 12 is made approximately the length of the femur plate 14 and the soft tissue is pulled apart so that the femur plate may be placed in position. Cancellous and cortical screws are then positioned over their respective openings in the femur plate 14 and secured to the femur 12.

When performing percutaneous surgery the skin of the thigh 8 is opened laterally near the knee and a transverse cut 92 is made and femur plate 14 is inserted at that opening and guided against the femur 12 proximally toward the hip. The proximal end of the femur plate 14 may include a contoured tip 84 to ease the percutaneous installation of the femur plate 14.

While the femur plate 14 may be made of any suitable size depending on the size of the human in which the plate is to be installed, the femoral plates 14 may be available in various lengths so that they will be available when trauma strikes. For example, the femoral plates may be provided with varying lengths including for example 5, 8, 11, 14 or 18 screw holes in the shaft.

The cortical and cancellous screws are manufactured of any suitable durable material that are typically manufactured of a wrought titanium alloy for example ASTM F-136 ELI.

Referring now to FIG. 12A, the femur plate 14 may further include a cannulated cancellous screw for positioning in the condylar portion 46 of the femur 12. The cannulated cancellous screw 62 preferably has a length slightly shorter than the length of the portion of the cancellous bone at the condylar portion 46 such that the cannulated cancellous screw does not contact the opposite cortical bone. The cannulated cancellous screw may, for example, be 8 millimeter cannulated cancellous and preferably as shown in FIG. 12A include external threads 48 located on proximal portion 38 of the cancellous screw 46. The external threads 48 mate with the internal threaded opening 49 of the distal portion 44 of the femur plate 14. The cannulated cancellous screw 62 provides additional structural support to the condylar portion 46 of the femur 12. Alternatively, the head of the cancellous screw 62 may be smooth, thereby allowing the head to spin in the plate without locking. The spinning achieves a lagging effect, i.e. drawing the fragments together.

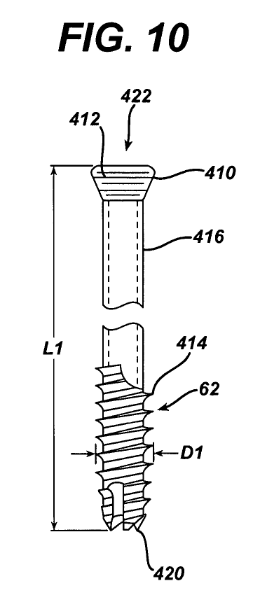

Referring now to FIG. 10, the cannulated cancellous screw 62 is shown in greater detail. The cannulated cancellous screw may not be in any particular size and may include a diameter D1 of, for example, 4 to 10 millimeters. The cannulated cancellous screw has a length L1 sufficient to occupy most of the condylar portion 46 of the femur or long bone 12. To provide for rigid attachment of the cannulated cancellous screw 62 to the bone plate 14, the cancellous screw 62 preferably includes a head 410 having external threads 412 which may mate with internal threaded opening 49 of the bone plate 14 (see FIG. 12A). The cannulated cancellous screw 62 includes external threads 414 and may include an unthreaded shank portion 416. The cannulated cancellous screw 62 may include a self-tapping tip 420 which may also serve as a self-drilling as well as a self-tapping tip. As shown in FIG. 10, the threads 412 on the head 410 are tapered to provide for a tight locking fit with the bone plate 14. The cannulated cancellous screw 62 is by definition cannulated or includes a central longitudinal opening 422.

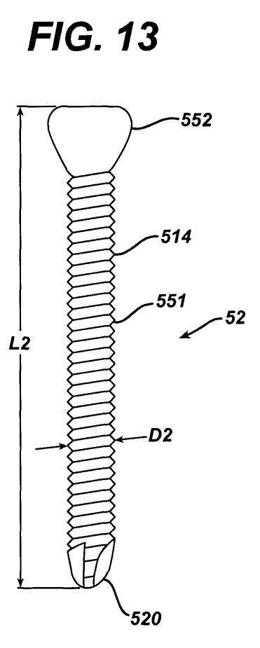

Referring now to FIG. 13 a cortical screw 52 is shown. The cortical screw 52 includes threads 514 which are adapted for securing cortical bone. The cortical screw 52 may include an unthreaded shank portion (not shown). The cortical screw 52 includes a head 552 which may, as shown in FIG. 13, have a generally oval shape. The cortical screw 52 may also include a self-tapping tip 520 which may also include self-drilling provisions. The cortical screw 52 has a length L2 which preferably is of sufficient length to engage the cortical bone on the opposite or exit side of the bone. The cortical screw 52 further includes a thread diameter D2 which is of sufficient size to provide sufficient holding power and engagement with the cortical bone. For example and as shown in FIG. 13, the cortical screw 52 has a diameter D2 of, for example, 3.5 to 6 millimeters.

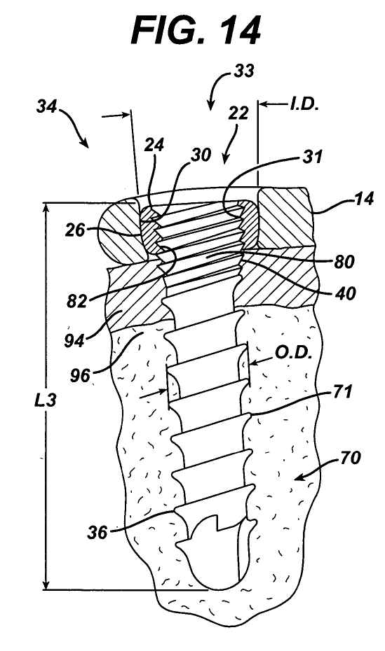

Referring now to FIG. 14 an attachment component according to the present invention is showed as cancellous screw 70. The screw 70 includes a distal portion 36 which has an outside diameter OD which is less than the inside diameter ID of the internal wall or surface 30 of the plate hole 22. The screw 70 further includes external threads 80 located on the proximal portion 40 of the screw 70. Preferably and as shown in FIG. 14, the external threads 80 are tapered. The external threads 80 are mateably engageable with the internal threads 82 on the bushing 24. The bushing 24 is pivotally engageable with the plate 14. The radially exterior surface 26 of the bushing 24 has a generally spherical shape and is mateably fitted with the interior wall or surface 30 of the plate hole 22. The interior threads 82 of the bushing 24 is larger than the outside diameter of cancellous threads 71 on the screw 70 to permit the distal portion of the 36 of the screw 70 to slidably pass or thread through the plate hole 22. The cancellous threads 71 are adapted for efficient engagement with cancellous bone 96 and the screw 70 has a length L3 which is sized to provide for the cancellous thread 71 to engage a significant portion of the cancellous bone 96.

Referring now to FIG. 18, a fully threaded cancellous screw 56 is shown for use with the bone plate 14. The cancellous screw 56 includes a head 610. The head 610 may have any suitable shape and may, for example, be flat head as shown in FIG. 18 or have a pan head shape. The screw 56 unlike cancellous screw 70 (see FIG. 6) does not include threads on the head of screw 56. The lack of screw threads on the head of screw 56 allows the head to spin on the bushing 24 without locking, thereby achieving a lagging effect. The cancellous screw 56 has a length L4 to provide for engagement with a suitable portion of the cancellous bone (not shown). The cancellous screw has threads 614 which are adapted for engagement with cancellous bone. The thread 614 has a diameter D4 which is sized for efficient and effective support and engagement with the cancellous bone. For example, the cancellous screw 56 may have a diameter D4 of, for example 2 to 6 millimeters. The cancellous screw 56 further includes a tip 620. The tip 620 may optionally include self-drilling and/or self-tapping features.

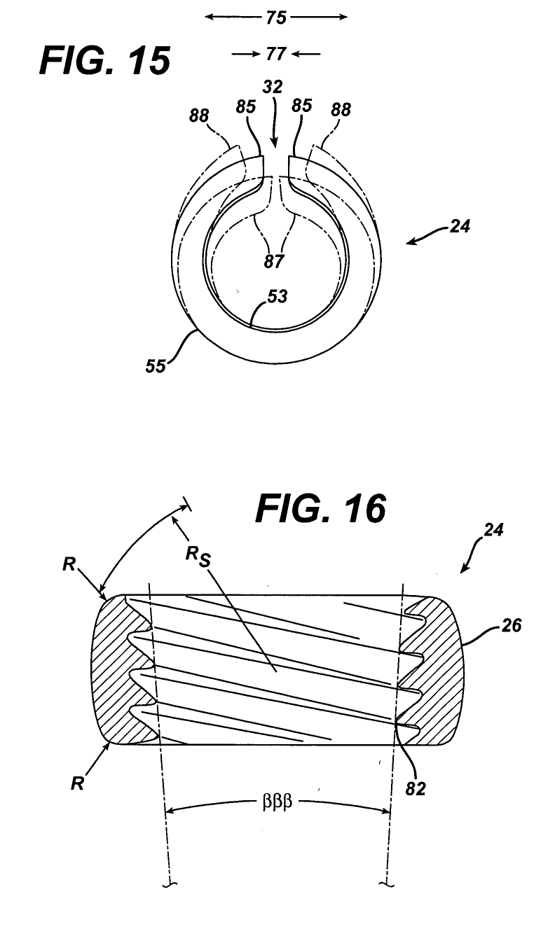

Referring now to FIG. 15, the bushing 24 is shown in greater detail. The bushing or collet is manufactured of any suitable durable material that is compatible with the human body. For example the collet may be made of cobalt chrome, stainless steel or titanium. For example the bushing 24 may be manufactured of a wrought titanium alloy. Such a wrought titanium alloy is ASTM F-136 ELI.

The bushing 24 preferably includes a radial opening or passageway 32 on the periphery of the bushing 24. The passageway 32 extends from the radially exterior surface 55 through the opposite radially interior surface 53. The bushing 24 has a first relaxed position 85 which represents the shape of the bushing 24 when not assembled into the plate 14. The bushing 24 further has an assembled position 87 as shown in the dotted line. The assembled position 87 represents when the bushing 24 is placed within the plate 14 and when the screws are not installed. The bushing 24 further has an expanded position 88 shown in phantom in which the bushing 24 is shown with the bushing 24 installed in the plate 24 and the screws installed within the bushing 24.

As can be seen in FIG. 15, the bushing 24 is contracted when the assembled position 87 to provide for an interference fit between the bushing 24 and the plate 14. Further as shown in FIG. 15, the bushing 24 is expanded as it moves from the assembled position 87 to the expanded position 88. This occurs because the tapered threads during engagement cause the bushing 24 to enlarge. The enlarging of the bushing 24 causes a tighter interference between the bushing 24 and the plate thereby securely locking the bushing in its polyaxial oriented position with minimal stress.

Referring now to FIG. 16 a cross-section of the bushing 24 is shown. As shown in FIG. 16 preferably the bushing 24 has a spherical radius R_{S} which defines the radial exterior surface 26 of the bushing 24. By providing a spherical radius R_{S} the bushing 24 may be oriented into a number of angular positions with respect to the plate.

Referring to FIG. 16 the internal threads 82 of the bushing 24 have a taper defined by an internal angle βββ. The angle βββ may be, for example, from 3 to 30 degrees. As shown in FIG. 16 the truncated spherical shape of the radial exterior surface 26 may be modified by corner radius R.

While the fracture repair system of the present invention includes the bushing to provide for positioning of the attachment component in a variety of diverging directions while providing for reduced stress at the plate, when percutaneously securing a bone screw to a bone plate location which does not provide for the pivotal securement of the bushing arrangement, it is critical that the screws in such fixed locations be properly positioned.

Referring now to FIG. 17 preferably the femur plate 14 is used in conjunction with drill guide 200. Drill guide 200 is installed onto the femur plate 14 during surgery and is utilized to guide drills and screwdrivers to properly orientate the screws that are placed in the proximal portion of the plate 14. The drill guide 200 includes a locating feature 202 in the form of, for example, an elongated pin which closely fits to the elongated slots of a plate. The drill guide includes a riser portion 204 and a bar portion 206 which is positioned parallel and spaced from the plate 14.

The bar portion 206 includes a series of bushing holes 210 which are in alignment with the center of the elongated openings 54 in the plate 14. To properly secure the drill guide 200 to plate 14, for example, the drill guide 200 may include a securing screw 214 which may be slidingly fitted to an opening 216 in the riser portion 204 and which may be secured to tapped opening 60 in the plate 14.

The drill guide 200 may be utilized both in conventional open surgery and in percutaneous surgery. When utilized in percutaneous surgery the bushing holes 210 may be utilized to guide trocars which will open the skin and tissue around the openings permitting the screws to be properly secured. Since the human anatomy is generally symmetrical, the drill guide 200 is either a right hand or left hand drill guide and the right hand and left-hand drill guide are different, but generally symmetrical with each other. It should be appreciated that the drill guide may be utilized for any bone plate for supporting any long bone for example a tibia, humerus, ulna, radius or fibula.

While heretofore the fracture repair system has been described in more detail as a femur plate, it should be appreciated that the plate may be utilized for supporting any long bone for example a tibia, humerus, ulna, radius or fibula.

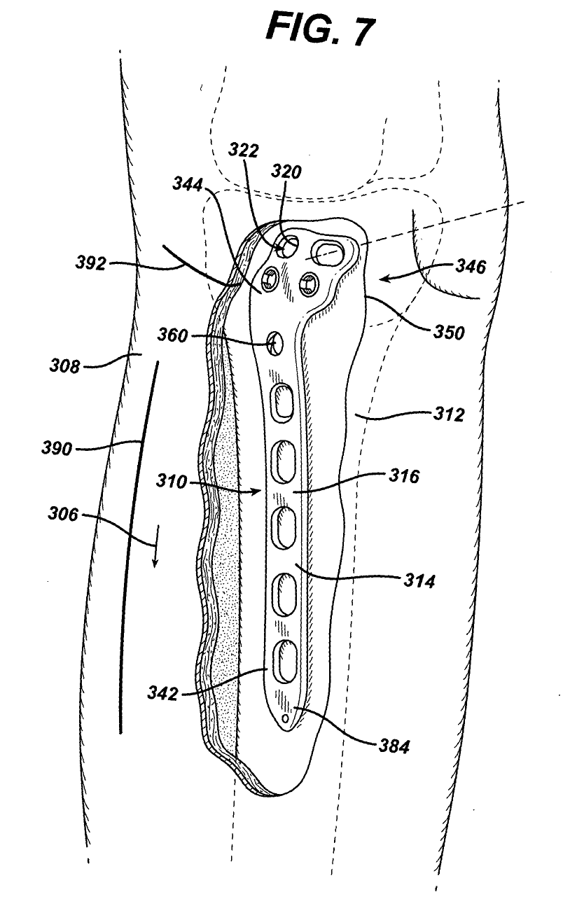

Referring now to FIGS. 7-9 and 11, a tibia plate 314 for installation onto a tibia 312 is shown. The fracture repair system 310 for use on the tibia 312 includes a tibia plate 314 having a body portion 316. The body portion 316 includes a distal portion 342 and a proximal portion 344. The tibia plate 314 like the femur plate 14 is preferably positioned laterally on the long bone. The lateral position of the tibia plate reduces the amount of soft tissue that must be dislocated to position the tibia plate 314. Since the human anatomy is generally symmetrical, the tibia plate 314 is either a right hand or left hand tibia plate and the right hand and left-hand tibia plates are different, but generally symmetrical with each other. The proximal portion 344 of the tibia plate 314 is designed to follow the general contours of the lateral proximal tibia. The proximal portion 344 of the plate 314 is contoured to fit the lateral condyle 350 of the condylar portion 346 of the tibia 312. The body portion 316 of the tibia plate 314 like the body portion 16 of the femur plate 14 has a generally arcuate cross-section to conform to the distal shaft of the tibia 312.

The tibia plate 314 like the femur plate 14 may be made of any suitable durable material that is compatible with the human immune system and may for example be made of a durable non-corrosive material such as stainless steel, cobalt chrome or titanium. For example, the tibia plate may be manufactured from a forged or wrought titanium alloy. For example, such a titanium alloy may be ASTM F-620-97 or ASTM F-136 ELI.

Referring now to FIG. 7, the tibia plate 314 may be inserted into the human anatomy percutaneously or by conventional open surgery. When inserted by conventional open surgery, the leg 308 is cut with a longitudinal incision 390 of length roughly equal to that of the tibia plate 314. The soft tissue is moved away from the tibia 312 and the tibia plate 314 is placed against the tibia 312. Screws such as those for the femur plate are utilized to secure the tibia plate 314 to the tibia 312. If the tibia plate 314 is to be inserted percutaneously, a smaller longitudinal incision 392 is made in the skin of the leg 308 near the knee and the distal portion 342 of the body portion 316 of the tibia plate 314 is inserted in the incision 392 in the direction of arrow 306 toward the distal portion of the leg. A contoured tip 384 on the distal portion of the 342 of the tibia plate of the tibia plate 314 is shaped to ease the insertion of the tibia plate along the contour of the tibia 312 in the direction of arrow 306.

For installation either percutaneously or by conventional open surgery of the tibia plate 314 drill guides (not shown) such as drill guide 200 for the femur plate as shown in FIG. 17 are utilized. Again, as with the femur plate, the drill guide may be utilized to guide the drill and the screws whether the plate and screws are inserted percutaneously or by conventional open surgery. It should be appreciated that a left-hand drill guide (not shown) and a right hand drill guide (not shown) are necessary respectively for the right hand and left-hand tibia plates (not shown).

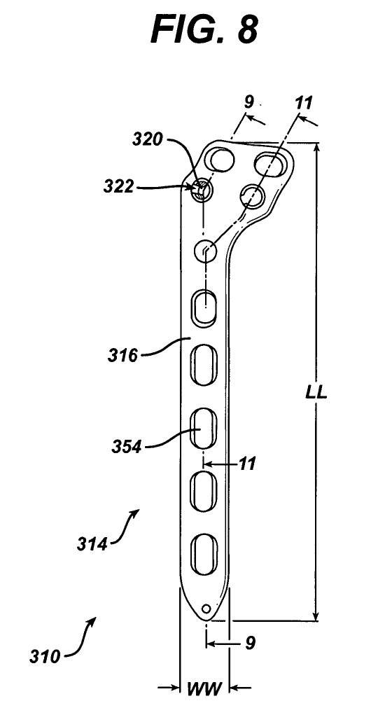

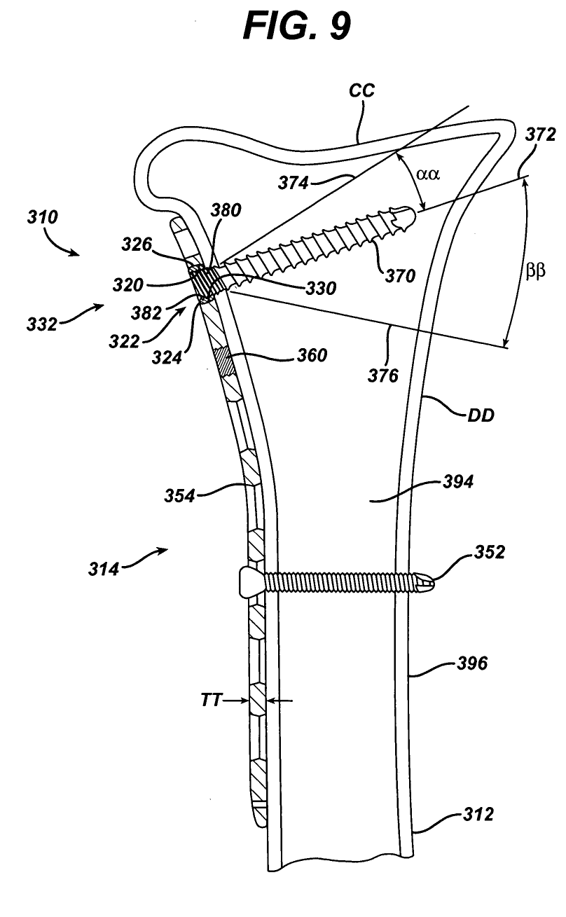

Referring now to FIG. 8, the tibial plate 314 may be made of sufficient dimensions to properly support the tibia 312. The proper dimensions of the tibial plate 314 are dependent thus on the size of the particular tibia to be treated as well as the inherent strength of the material from which the tibial plate 314 is made. For example, the tibial plate 314, if made of titanium, may have a thickness TT (see FIG. 9) of, for example, approximately 1/16 to 1/14 of an inch and a WW width of around ¼ to ¾ inch and a length LL of, for example, from 5-10 inches. To provide for a range of standard tibial plates, the tibial plates may be provided in varying lengths of, for example, a length with a number of elongated openings 354 of, for example, 4, 7, 11, or 14 elongated openings.

According to the present invention and referring to FIGS. 7-9 and 11, the tibial plate 314 includes the body portion 316 which conforms at least partially to the contour of the tibia 312. The tibia plate 314 also includes an interior wall 320 which defines a tibia plate hole 322 through the body portion 316.

Referring to FIG. 9, the tibial plate 314 further includes one or more bushings 324. The bushing 324 includes a radially exterior surface 326 and an opposite radially interior surface 330. The opposite radially interior surface 330 defines a passageway 332 there through. The exterior surface 326 of the bushing 324 and the interior wall 320 (see FIG. 9) of the plate 314 cooperate with each other and are configured to permit polyaxial rotation of the bushing 324 within the plate hole 322. The tibial plate 314 further includes an attachment component 370 in the form of, for example, a cancellous screw. The screw 370 includes a distal portion 336 sized for clearance passage through the passageway 332 and into the cancellous bone 394.

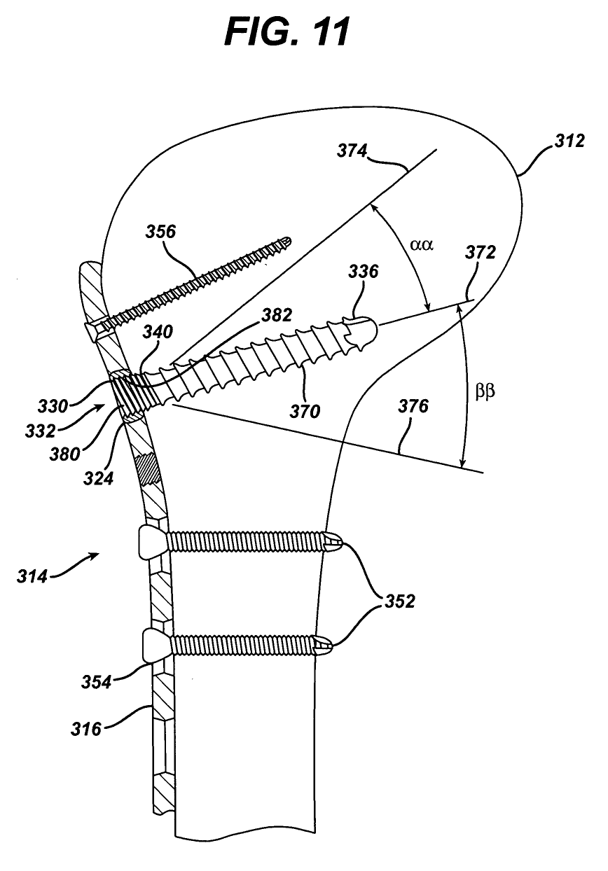

The screw 370 further includes a proximate portion 340 sized to press the bushing 324 against the inner wall or surface 330 of the plate 324 to form a friction lock between the bushing 324 and the plate 314 in a selected polyaxial position. For example, the cancellous screw 370 may be in a first polyaxial position 372 as shown in solid line 372 (see FIG. 11). Alternatively, the cancellous screw 370 may be oriented an angle αα from the first position 372 into a second position 374 as shown in phantom. Alternatively, the cancellous screw 370 maybe positioned in, for example, a third position 376 positioned at an angle ββ from the first position 372. The cancellous screw 370 may thus be positioned with a diverging angle αα or ββ from the first position 372.

Preferably and as shown in FIG. 11, the proximal portion 340 of the cancellous screw 370 includes external tapered threads 380 which mate with internal threads 382 located within the bushing 324. By providing tapered threads as the cancellous screw 370 is screwed into the bushing 324, the bushing 324 expands with the radially exterior surface 326 of the bushing, seating and securing against the radially interior surface 330 of the plate 314. This provides for stress-free, secure locking of the screw 370 to the plate 314.

Alternatively, the attachment component which mates with the bushing 324 may be provided without any threads in the proximal portion of the attachment component similarly to the screw 170 of FIG. 6A. Such a screw, for example screw 56 of FIG. 6 or screw 980 of FIG. 25, will provide for polyaxial positioning of the attachment component with reduced stress. The screw 56 or 980 unlike cancellous screw 70 (see FIG. 6) does not include threads on the head of screw 56. The lack of screw threads on the head of screw 56 or 980 allows the head to spin on the bushing 24 without locking, thereby achieving a lagging effect.

By positioning the cancellous screw 370 into the first position 372 or the second position 374 or the third position 376, the screw 370 maybe positioned to properly secure fragments. For example as shown in FIG. 9 the cancellous screw 370 being positioned in second position 374 may provide for the securing of a fragment CC while the positioning of the cancellous screw 370 in the third position 376 may provide for the securing of fragment DD.

The fracture repair system 310 for use for repairing a fractured tibia may include additional attachment components such as additional attachment component 370. Thus the fracture repair system may include a second cancellous screw 370 positioned at a second plate hole (not shown). In addition to a plurality of cancellous screws 370, the fracture repair system 310 may include, in addition to the polyaxial screws, additional cancellous or cortical screws. For example, Referring to FIGS. 9 and 11, the repair system 310 may include fully threaded cortical screws 352 similar to the cortical screws 52 of the femur plate 14. The cortical screws 352 preferably extend through the cancellous bone 394 and engage with the cortical bone 396. The fracture repair system 310 may further include cancellous screws for example cancellous screws 356 located in the proximal portion 344 of the boneplate 314 as shown in FIG. 11. Such cancellous screws 356 are preferably of a length short enough that they do not reach through to the opposed cortical bone 396. The tibial plate 314 may include one or more tapped openings 360 in the tibial plate 314 which may be utilized to secure a drill guide (not shown), similar to the drill guide 200, for aligning a drill and a screw driver for the insertion of the screws 352 into the tibia.

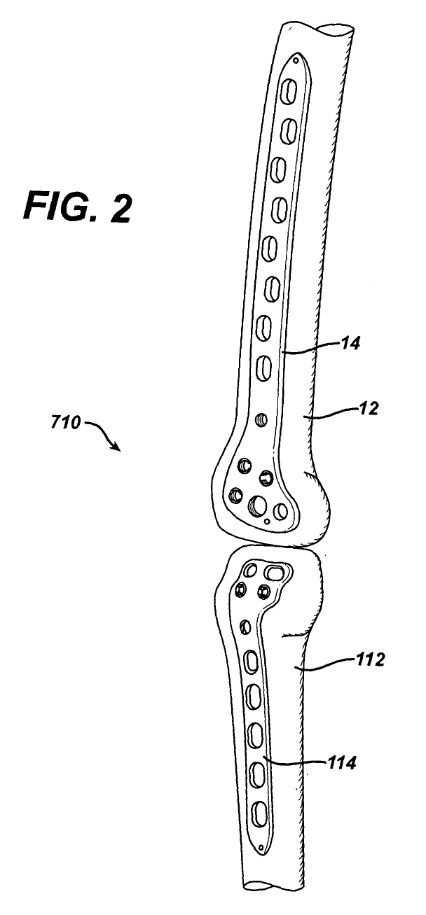

Referring now to FIGS. 1 and 2 a fracture repair system 710 is shown. The fracture repair system 710 comprises an assembly of both a femur plate 14 and a tibia plate 114. Frequently the polyaxial plates of the present invention are sold as a fracture repair system 710 including both a tibial plate 114 and a femur plate 14. Such a combination is often required in severe knee trauma caused, for example, in front-end auto accidents. It should be appreciated that a fracture repair system may include a plate for any other long bone for example a humerus, ulna, fibula or radius.

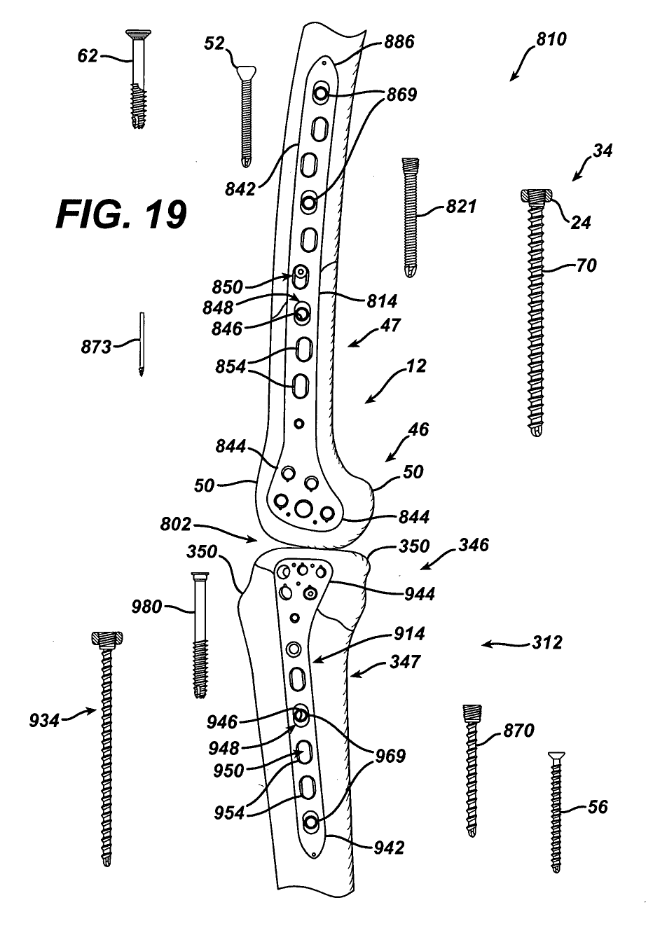

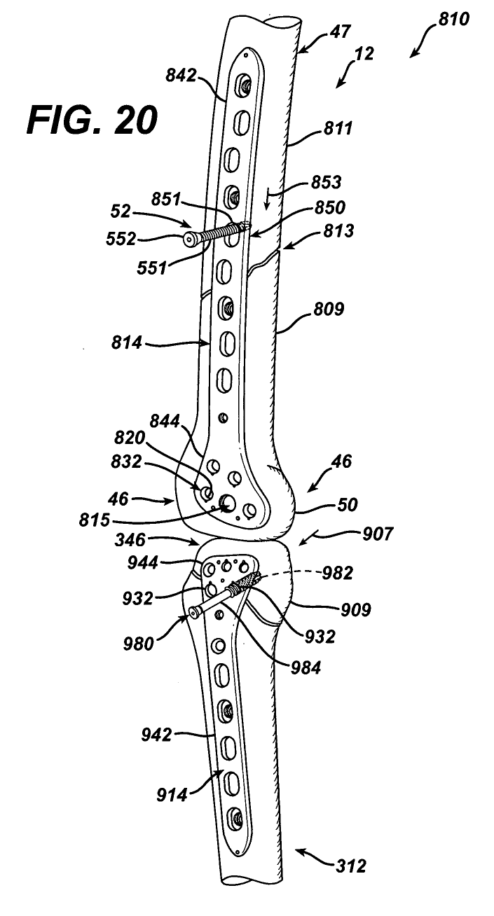

According to the present invention, referring now to FIG. 19 and FIG. 20, another embodiment of the present invention is shown as joint fracture system 810. The joint fracture system 810 is for use with a joint, for example, knee joint 802. The knee joint 802 is associated with adjoining first and second long bone, for example, the femur 12 and the tibia 312. The joint fracture system 810 includes a first plate 814. The first plate 814 cooperates with, for example, the first long bone 12. As shown in FIG. 19, the first long bone 12 may be in the form of a femur. It should be appreciated that the long bone 12 may alternatively be, for example, a tibia, a fibula, a humerus, a radius or an ulna. First plate 812 includes a first plate head portion 844 and a first plate body portion 842. The first plate body portion 842 has an internal wall 846 defining a first plate first body hole 848. The first plate body portion 842 further defines a first plate second body hole 850 spaced from the first plate first body hole 848. Joint fracture system 810 further includes a first plate rigid body attachment component 821, including a stem portion 823 for passage through the first plate first body hole 848 and into the bone 12. The first plate rigid body attachment component 821 further includes an opposed cap portion 825 adapted to rigidly cooperate with the first plate 814 at, for example, the first plate first body hole 848.

The joint fracture system 810 further includes a first plate movable body attachment component in the form of, for example, a solid, fully threaded, cortical screw 52. The first plate movable body attachment component 52 includes a stem portion 551 for passage through the first plate second body hole 850 and into the bone 12. The first plate movable body attachment component 52 further includes an opposed cap portion 552 adapted to movably cooperate with the first plate 814. The screw 52 is shown in greater detail in FIG. 13.

The joint fracture system 810 further includes a second plate 914 for cooperation with the second long bone 312. The second plate 914 may be in the form of, for example, a tibia plate and may cooperate with a long bone in the form of, for example, tibia 312. The second plate 914 includes a second plate head portion 944 and a second plate body portion 942. The second plate body portion 942 has an internal wall 946 defining a second plate first body hole 948 and a spaced apart second plate second body hole 950 there through.

The joint fracture system 810 further includes a second plate rigid body attachment component in the form of, for example, attachment component 821. The second plate rigid body attachment component 821 may be identical to the first plate rigid body attachment component 821. Therefore, the second plate rigid body attachment component 821 includes the stem portion 823 for passage through the second plate first body hole 948 and into the bone 312 and the opposed cap portion 825 adapted to rigidly cooperate with the second plate 914.

The joint fracture system 810 may further include a second plate movable body attachment component in the form of, for example, component 52 including the stem portion 551 for passage through the second plate second body hole 950 and into the bone 312 and the opposed cap portion 552 adapted to movably cooperate with the second plate 312.

Referring now to FIG. 20, femur plate 814 is shown in position on long bone or femur 12 and shown for use in repairing a transverse fracture 813. To repair the transverse fracture 813, one end, for example, head portion 844 of the plate 814 is secured to, for example, condylar portion 46 of the femur 12. The head portion 844 may be secured to the condylar portion 46 of femur 12 with, for example, cannulated cancellous screw 62 (see FIG. 12A) which may be secured to large hole 815 in the head portion 844 of the plate 814. A screw, for example, the movable body attachment component, cortical screw 52 (see FIG. 13), is fitted into second hole 850 of the plate 814 with stem 551 of the screw 52 positioned against proximal edge 851 of the hole 850. The screw 52 is then threaded into the bone 12 until the head or cap 552 of the cortical screw 52 contacts the proximal edge 851 of the plate 814.

When the cap 552 of the cortical screw 52 contacts the proximal edge 851 of the plate 814, the plate 814 urges the screw 52 in the direction of arrow 853, which in turn urges first or proximal fragment 811 of the femur 12 in the direction of arrow 853 thereby moving the proximal fragment 811 of femur 12 in contact with second or distal fragment 809 of the femur 12. Thus, the cortical screw 52 cooperating with the plate 814 urges the fragments 811 and 809 into contact with each other. With the fragments 809 and 811 in firm contact with each other, blood flow within the long bone 12 and healing of the fracture site is facilitated.

Continuing to refer to FIG. 20, the use of the present invention to join bone fragments in the condylar portion 346 of long bone 312 is shown. For example, a bone fragment 909 is shown separated from the condylar portion 346 of long bone, for example, tibia 312. A screw, for example, a lag screw such as a partially threaded cancellous screw 980 ( see FIG. 25) may be inserted in, for example, large polyaxial opening 932 in the head portion 944 of the plate 914 and screwed into the condylar portion 346 of the tibia 314. As the screw 980 is advanced in the condylar portion 346 of tibia 312, the screw 980 may contact the fragment 909. As threaded portion 982 of the screw 980 contacts the fragment 909 and as the condylar portion 346 of the tibia 314 is in cooperation with relief portion 984 of the screw 980, the fragment 909 is urged by the screw 980 in the direction of arrow 907 until the fragment 909 moves from its position shown in phantom to the position shown in solid in full contact with the condylar portion 346 of the tibia 314.

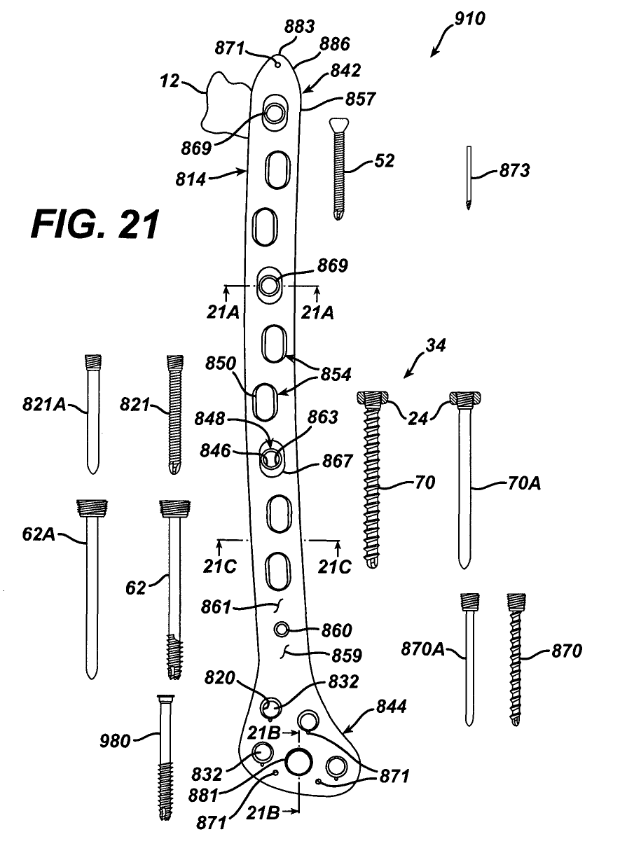

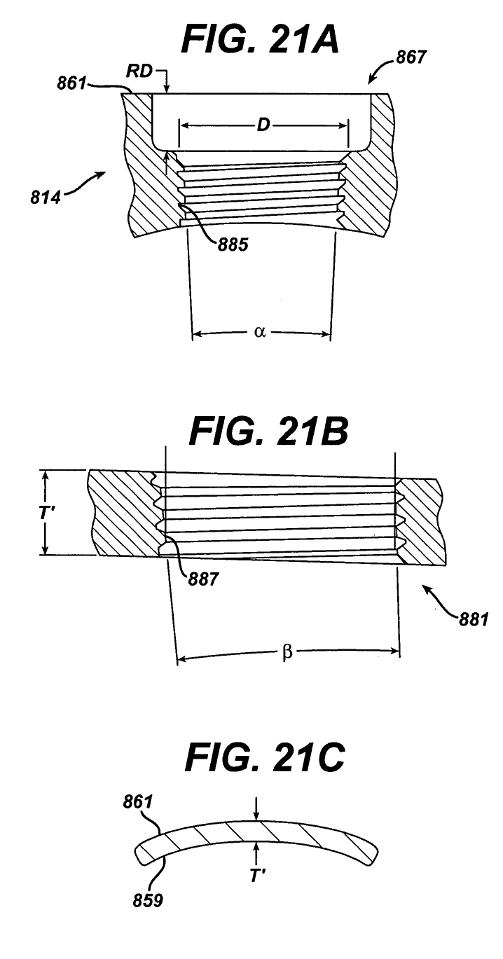

Referring now to FIG. 21, another embodiment of the present invention is shown as fracture repair system 910. The fracture repair system 910 is used for engagement with a bone, for example, the long bone or femur 12. The femur 12 may include a condylar portion 46 and a shaft portion 47 (see FIG. 20). The fracture repair system 910 includes a plate, for example, long bone plate or femur plate 814. The plate 814 includes a head portion, for example, head portion 844 and a body portion, for example, body portion 842. The head portion 844 includes an internal wall 820 which defines a head hole or passageway 832 for the plate 814. The head portion 844 is adapted for cooperation with the condylar portion 46 of the femur 12 (see FIG. 19). The body portion 842 includes internal wall 846 defining body hole 848 through the plate 814.

The fracture repair system 910 further includes a bushing, for example, bushing 24 (see FIG. 14). The bushing 24 includes a generally spherically exterior surface 26 adapted for cooperation with the head hole 832 of plate 814. The bushing 24 further includes an opposed interior surface 31 defining a passageway 33 through the bushing 24. The exterior surface 26 of the bushing 24 and the head hole 32 of the plate 814 are configured to permit polyaxial rotation of the bushing 24 within the head hole 832.

The fracture repair system 910 further includes a head attachment component, for example a polyaxial, rigid, cancellous screw assembly such as screw assembly 34 (see FIG. 14). The screw assembly 34 includes a distal portion 36 sized for clearance passage through the passageways 32 and 33 and into the bone 12. The head attachment component 34 further includes an opposed proximal portion 40 sized to urge the bushing 24 against the internal wall 820 of the plate 814 to form a friction lock between the bushing 24 and the plate 814 in a selected polyaxial position. The head attachment component 34 is positionable in an orientation extending divergently from the plate 814.

The fracture repair system 810 further includes a first body attachment component, for example, a rigid cancellous screw such as screw 821 including a stem portion 823 for passage through the first body hole 848 and into the bone 12. The first body attachment component 821 further includes an opposed cap portion 825 sized to cooperate with the plate 814.

It should be appreciated that the plate 814 of the system 910 of FIG. 21 may have a shape and configuration generally similar to that of the femur plate 14 of FIGS. 3, 4 and 5. For example, the plate 814 may have an outer periphery 857 which is substantially the same as outer periphery of the femur plate 14 of FIGS. 3, 4 and 5. As shown in FIG. 21, the plate 814 may define a bone contact surface 859 which closely conforms to the bone or femur 12. The plate 814 may thus have a generally arcuate cross-section as shown in FIG. 21(c) and have an outer surface 861 which is generally parallel and spaced from the bone contact surface 859. Surfaces 859 and 861 may be spaced apart, for example, a thickness T¹ which may be similar to the thickness T of the plate 14 of FIG. 6.

As shown in FIG. 21, the fracture repair system 910 may include the second body hole 850 through the body portion 842 of the plate 814. The fracture repair system 910 may further include the second body attachment component in the form of, for example, the cortical screw 52 (see FIG. 13). The cortical screw 52 includes the stem portion 551 for passage through the second body hole 850 and into the bone 12 and an opposed cap portion or head 552 sized to cooperate with the plate 814. While the second body hole 850 may have any suitable shape, as shown in FIG. 21, the second body hole 850 may be in the form of an elongated opening 854 similar to the elongated openings 54 of the plate 14 (see FIG. 4).

As shown in FIG. 21, when the plate 814 has a length substantially greater than the width of the plate 814, a plurality of elongated openings 854 may be provided on the body portion 842 of the plate 814. As shown in FIG. 21, the cortical screw 52 and the elongated openings 854 provide for movable attachment of the plate 814 to the bone 12.

The first body hole 848 may have any suitable shape for receiving the first body attachment component or screw 821. For example and as shown in FIG. 21, the screw 821 may be in the form of a screw capable of fixed attachment to the plate 814. The fixable attachment of the screw 821 to the plate 814 may be accomplished by, for example, internal threads 863 formed in wall 846 of the plate 814, which cooperate with external threads 865 formed on cap portion 825 of the screw 821. The internal thread 863 and the external threads 865 may, as shown in FIG. 21, be tapered to provide for rigid locking of the screw 821 to the plate 814.

As shown in FIG. 21, the plate 814 may include elongated recesses 867 positioned centrally about the body hole 848. The elongated recess 867 may, for example, have a shape substantially the same as the elongated openings 854. For example, the elongated openings have a width WF1 and a length LF1 which are substantially the same as the width WF2 and length LF2 of the elongated recesses 867. The elongated openings 854 form a first location feature for cooperating with the drill jig 200 of FIG. 17. Similarly, the elongated recesses 867 define a second location feature for cooperating with the drill jig of FIG. 17. By making the first location feature and the second location feature for the jig 200 be substantially identical in the plate 814, the drill jig 200 for use on the plate 14 of FIGS. 2 through 5 may also be used for the plate 814 of FIG. 21.

While the plate 814 may have a solitary first body hole 848, the plate 814 preferably includes a plurality of the first body holes because the plate 814 has a length substantially greater than its width. For example and as shown in FIG. 21, the plate 814 may include additional threaded body holes 869 which are similar to the first body hole 848.

To permit the plate 814 to be used with body fixed screws 821 and the body movable screws 52, the body portion 842 of the plate 814 may include a pattern of elongated openings 854 and threaded body holes 869. As shown in FIG. 21, the threaded body holes 869 are centrally located along the body portion 842 of the plate 814. Between each adjoining threaded body hole 869, for example, a pair of spaced apart elongated openings 854 are positioned. The plate as shown in FIG. 21 includes six elongated openings 854 and three threaded holes 869 forming a total of nine body mounting holes.

To accommodate a wide range of patient femur sizes and shapes, it should be appreciated that the plate 814 may be provided with a different number of body mounting holes. For example, in addition to the nine hole configuration as shown in FIG. 21, the plates may be provided with six, twelve, fifteen or eighteen mounting holes in the body. While the threaded body holes 869 may be centrally positioned on the plate 814, the elongated openings 854 may be offset from the center of the plate and be formed in a staggered position to as shown in FIG. 21 provide a variety of mounting positions for the plate.

To provide for percutaneous installation of the mounting plate 814, the plate 814 may include a threaded mounting opening 860 for mounting the plate 814 to the drill guide 200 (see FIG. 17).

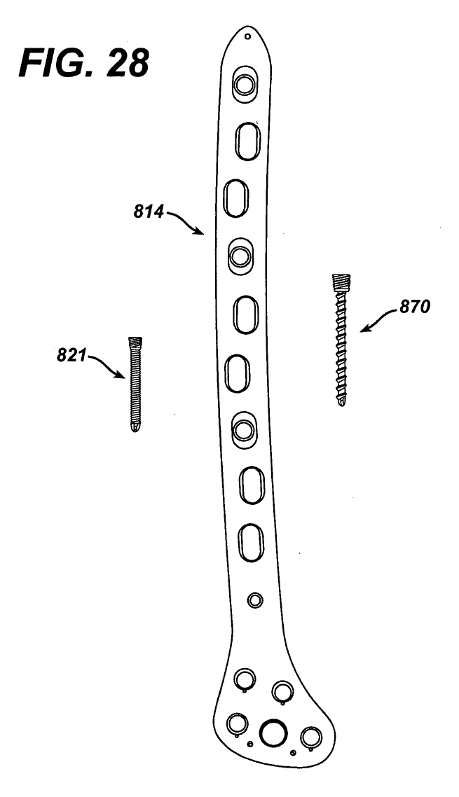

To assist in positioning the plate 814 in a proper position relative to the femur 12, the plate 814 may include a plurality of k-wire holes 871. The k-wire holes 871 are for use with k-wires 873 (see FIG. 28). The plate 814 may be positioned visually over the bone or femur 12 and the k-wires 873 may be installed through k-wire holes 871 into the femur 12. The k-wire holes 871 may be positioned in the head portion 844 and in the proximal portion 842. The K-wire hole 871 in the proximal portion 842 may be used with a suture to move the plate 814 percutaneously along the bone 12.

As shown in FIG. 21, the plate 814 may include a plurality of spaced apart passageways 832 for use with the bushing 24 and the polyaxial cancellous screw 34. For example, as shown in FIG. 21, the plate 814 may include four spaced apart passageways 832.

As can be seen in FIG. 21, the plate 814 may be used with a wide variety of attachment components or screws. It should be appreciated that any connector or fastener which may be fitted into an opening in the plate 814 may be used within the discretion of the surgeon. The plate 814 as shown in FIG. 21 is particularly well suited for the use of particular fasteners or screws in particular openings in the plate 814. For example and as shown in FIG. 21, the elongated openings 854 are particularly well suited for use with the cortical screw 52. It should be appreciated that a lag screw, for example, a partially threaded cancellous screw (not shown) has a threaded stem such as screw 980 (see FIG. 25), may also be used in the elongated openings 854. The lag screw serves to adjoin bone portions from an axial fracture. The lag screw may include a relief portion of stem for clearance passage through the elongated opening 854 and a cap for cooperation with the plate 814.

The threaded body holes 869 are suited particularly for the cortical locking screw 821 (see FIG. 23). The locking screw 821 provides for rigid attachment of the screw 821 to the plate 814. The cortical locking screw 821 is particularly suited for patients with thin-shell or osteoporotic bone.

Occasionally, particularly in osteoporotic bone, bone adjacent the stem portion 823 of the screw 821 may become stripped in the bone 12. A locking cancellous screw 870 is particularly well suited for application in the threaded body holes 869 when the bone adjacent the locking cortical screw 821 is stripped. The screw 821 may be removed from the bone 12 and the screw 870 inserted into the plate 12 in its place. The screw 870 includes a threaded stem portion 873 which may include cancellous screw threads which may be less prone to stripping bone than the cortical threads of the stem portion 873 of the screw 870. As shown in FIG. 21, the threads 865 of the screw 821 may preferably be identical to the threads 877 of the screw 870 so that the screws 821 and 870 are interchangeable at the threaded body holes 869.

Passageways 832 in the head portion 844 of the plate 814 are particularly well suited for use with the attachment component or polyaxial screw assembly 34 (see FIG. 14). The attachment component 34 provides for the polyaxial positioning of the screw assembly 34. The screw assembly 34 may, for example, include the fully threaded cancellous screw 70 including threads for securing cancellous components or fragments of the condylar portion of the long bone. Alternatively, the passageways 832 are also compatible with the lag screw 980 (see FIG. 25). The lag screw 980 is particularly well-suited if the fragments of the condylar portion of the femur 12 are separated and need to be drawn together.

The plate 814 may further include a large threaded head hole 881 for which cannulated cancellous screw 62 (see FIG. 12A) may be used. The cannulated cancellous screw 62 is particularly well suited for joining the fragments in the condylar portion of the long bone 12 .

While the arrangement of the elongated openings 854 and the threaded plate body holes 869 may be arranged in any suitable order, the applicants have found that a threaded body hole 869 positioned opposed to the head portion 844,for example, at opposed end 883 of the plate 814 may be preferred. The end 883 of plate 814 will then be rigidly secured to the femur 12 and will avoid movement between the end 883 of the body portion 842 of the plate 814 and the long bone 12 as the patient walks. When all body holes are not used with screws, the end hole is preferably chosen as a screw location to provide stable support for the plate. A threaded body hole adjacent the end 883 permits the end of the plate 814 to be either rigidly or moveably secured.

When utilized for percutaneous installation, the plate 814 of the fracture repair system 910 may include a bullet nose 886. The bullet nose 886 has a bullet or tapered shape to assist in percutaneous insertion of the plate by the implanting surgeon adjacent the femur or long bone 12.

Referring now to FIG. 21A the threaded body hole 869 of the plate 814 is shown in greater detail. The threaded body holes 869 include internal threads 885 which mate with, for example, threads 865 of the screw 821 (see FIG. 23). Threads 885 may be tapered and defined by an included angle α. The threaded hole 869 has a diameter D selected to mate with the cap portion 825 of the screw 821 (see FIG. 23). The threaded body hole 869 is in alignment with the elongated recess 867. The elongated recess 867 is recessed a distance RD from outer surface 861 of the plate 814. In order to provide to sufficient strength in the threads 885, the threads 885 may, for example, be triple lead threads. In a triple lead thread, the screw advances axially as it is rotated three times as far as the distance between adjacent threads. By utilizing the triple lead threads for threads 855 while maintaining a single lead thread on the stem portion of the screw, for example, stem 823 of the screw 821 (see FIG. 23), a coarse cancellous thread may be used on the screw stem 823 and strong, fine threads may be used in the holes of the plate 814.

Referring now to FIG. 21B, the large threaded hole 881 is shown in greater detail. The large threaded hole 881 may include threads 887 which may be tapered and defined by an included angle β. The threads 887 like the threads 885 may be triple lead threads to provide for a strong thread in the plate 814 and in the cap portion of the screw while providing a coarse thread in the stem portion of the screw.

The plate 814 may be made of any suitable durable material that is biologically compatible with the human anatomy and preferable made of a high strength metal. For example, the plate may be made of stainless steel, cobalt chrome or titanium. Preferably the plate 814 is manufactured from a forged or wrought titanium alloy. One such suitable alloy is ASTM F-620-97 and another suitable alloy is ASTM F-136 ELI.

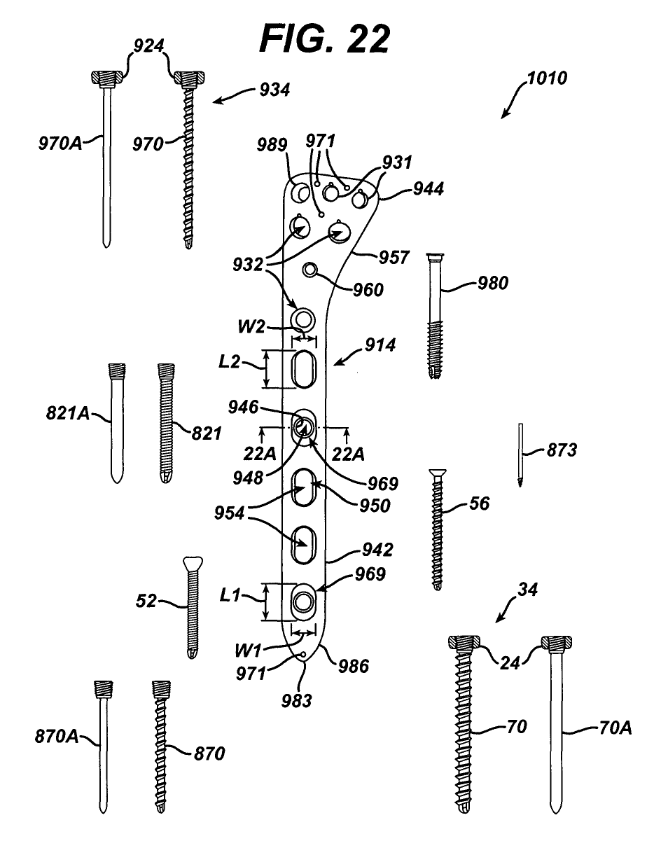

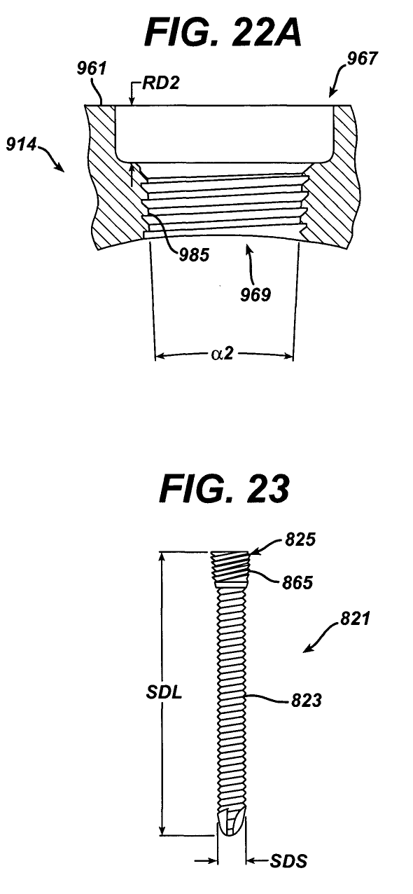

Referring now to FIG. 22, another embodiment of the present invention is shown as fracture repair system 1010. The fracture repair system 1010 is for use for engagement with a bone, for example a tibia 312 having a condylar portion 346 and a shaft portion 347 (see FIG. 19). The fracture repair system 1010 includes a plate in the form of for example a tibia plate 914. The tibia 914 may have any suitable size and shape and includes a head portion 944 and a body portion 942. The body portion 942 has an internal wall 946 defining a first body hole 948. The body portion 942 further includes a spaced apart second body hole 950 through the body portion 942.

The fracture repair system 1010 further includes a rigid body attachment component in the form of, for example, rigid cortical screw 821 (see FIG. 23) including a stem portion 823 for passage through the body hole 948 and into the bone 312 and an opposed cap portion 825 adapted to rigidly cooperate with the plate 814. The fracture repair system 1010 further includes a movable body attachment component in the form of, for example, moveable cortical screw 52 (see FIG. 13) including the stem portion 551 for passage through the second body hole 950 and into the bone 312. The movable body attachment component 551 further includes an opposed cap portion 552 adapted to movably cooperate with the plate 914.

The fracture repair system 1010 of FIG. 22 is particularly well adapted for use with bone when the bone is, for example, a tibia 312. The tibia 312 may either be osteoporotic or healthy bone. The choice of the use of locking and non-locking plate construction may depend on whether the bone is osteoporotic or healthy. If, for example, the bone is osteoporotic, a rigid attachment of the screws to the plate may be preferred. In such a rigid attachment, the rigid body attachment components in the form of, for example, rigid cancellous screw 821 (see FIG. 23) are utilized in the hole 948. Conversely, if the bone is not osteoporotic, the movable body attachment components, for example movable cortical screws 52 are utilized in the hole 950 for movable attachment with the plate 914.

While the plate 914 may have any suitable shape for cooperation with the long bone, for example, the tibia, the plate 914 may have an outer periphery 957 similar to the periphery of the tibia plate 314 of FIGS. 7, 8 and 9. The plate 914 may include additional threaded body holes 969 similar to the hole 948. Similarly, the plate 914 may include additional clearance holes similar to the hole 950 in the form of, for example, elongated openings 954. Similar to the configuration of the femur plate 814, the tibia plate 914 may include elongated recesses 967 centrally located around the threaded body holes 967. The elongated recesses 967 have a width W1 and a length L1 preferably identical to the width W2 and the length L2 of the elongated openings 954. The elongated openings 954 and the elongated recesses 967 are preferably sized for compatibility with the drill guide 200 of FIG. 17.

Elongated opening 954 and the elongated recesses 969 may, as shown in FIG. 22, be centrally located along the length of the body 942. As shown in FIG. 22, two elongated openings 954 may be positioned between each threaded body hole 969. As shown in FIG. 22, two threaded body holes 969 and three elongated opening 954 representing a total of five openings are shown. The plate 914 may include any multiple of three openings, for example, 8, 11 or 14 openings.

Similarly to the plate 814 of FIG. 21, the plate 914 may include one of the threaded location holes 969 positioned at end 983 of the plate 914 to rigidly secure the end of 983 to the bone 312 and to prevent motion between the end 983 and the bone 312 as the patient walks. Further, since the end hole should be used whenever possible, even if all holes are not used, the use of a threaded body hole adjacent the end 883 permits the end of the plate to be either rigidly or moveably secured. Similarly, the plate 914 may include a bullet nose 986 similar to the bullet nose 886 of the plate 914. The plate 914 may include a threaded guide hole 960 for cooperation with the drill guide 200.

The plate 914 may further include a plurality of k-wire holes 971 for cooperation with the k-wire 973 of FIG. 28. As shown in FIG. 22, the plate 914 may include three k-wire holes 971 in the head 944 and one k-wire hole 971 adjacent the end 983 of the plate 914. The plate 914 may further include large passageways 932 similar to the passageways 832 of the plate 814 of FIG. 19 for cooperation with the attachment component, for example, screw assembly 34 (see FIG. 14). The attachment component 34 provides for polyaxial location of the cannulated cancellous screws 70.

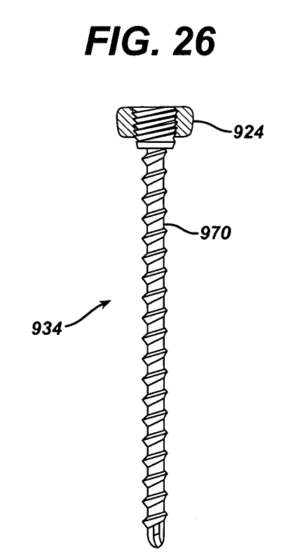

The plate 914 may further include small passageways 931 smaller than the passageways 932 for cooperation with polyaxial attachment component 934. The polyaxial attachment component 934 (see FIG. 26) is similar but smaller than the polyaxial attachment component 34 of FIG. 14.

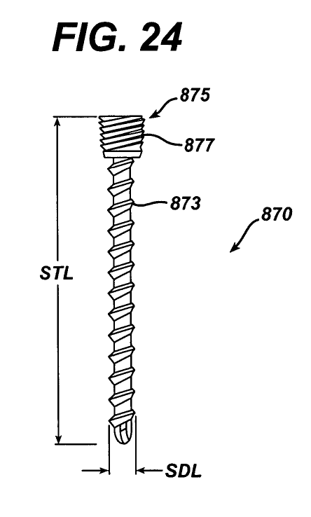

While it should be appreciated that any fastener which may fit in an opening in the plate may be utilized therewith, the plate 914 of FIG. 22 includes holes which are designed for use with particular fasteners. For example, as shown in FIG. 22, the elongated openings 954 are compatible with the cortical screws 52 of FIG. 13. It should be appreciated that the lag screw (not shown) having a stem like that of screw 980 of FIG. 25 may similarly be put in the elongated openings 954. The lag screw may be used to adjoin portions of bone in an axial fracture. The threaded body openings 969 are compatible with the body attachment component or screw 821 of FIG. 23 and with the cancellous screw 870 of FIG. 24. The screws 821 and 870 are chosen for use with plate 984 for the same reason that they are chosen for use with plate 814 of FIG. 21. The passageways 932 are compatible with screw assembly 34 including cancellous screw 70 of FIG. 14 as well as with the lag screw 890 of FIG. 25. The small passageways 931 are compatible with screw assembly 934 including screw 970 (see FIG. 26).

As shown in FIG. 22, the plate 914 may further include a small screw opening 989 located in the head portion 944 of the plate 914. The small screw opening 989 is designed for use with fully threaded cancellous screw 56 of FIG. 18.

Referring now to FIG. 22A the threaded body opening 969 is shown in greater detail. The threaded body opening 969 includes internal threads 985 which may, as shown in FIG. 22A, be similar to the internal threads 885 of the threaded body opening 869 of FIG. 21A and thus may be of a triple lead type. The threaded body opening 969 is centrally positioned with respect to the elongated recess 967. The elongated recess 967 is recessed from the surface 961 of the plate 914 a distance of RD2 which may be the same as distance RD of FIG. 21A. The threads 985 may be tapered and defined by an included angle α2 which may be identical to the angle α of the threads 885 of the plate 814 of FIG. 21A.

The plate 914 may be made of any suitable durable material that is biologically compatible with the human anatomy and preferable made of a high strength metal. For example, the plate may be made of stainless steel, cobalt chrome or titanium. Preferably the plate 914 is manufactured from a forged or wrought titanium alloy. One such suitable alloy is ASTM F-620-97 and another suitable alloy is ASTM F-136 ELI.

Referring now to FIG. 23, the screw 821 is shown in greater detail. The screw 821 as shown in FIG. 23 is a fully threaded cortical type screw with a fixed locking style. The screw 821 includes a stem 823 including cortical threads that extend to the cap 825 of the screw 821. The cap 825 includes tapered triple lead threads 865. As shown in FIG. 23, the screw 821 is self-tapping. Screw 821 may have various lengths SDL, for example, from 14 mm to 40 mm and may have a stem diameter SDS of, for example, 4.5 mm and a cap diameter of, for example, 5.5 mm.

Referring now to FIG. 24, the screw 870 is shown in greater detail. The screw 870 is similar to cancellous screw 70 of FIG. 14 and differs from the cancellous screw 70 of FIG. 14 only in its overall length. The screw 870 has, for example, a stem 873 having fully threaded cancellous threads. Screw 870 further includes a cap 875 having tapered external threads 877 similar to the threads 865 of the screw 821 (see FIG. 23) and therefore includes screw threads that are triple lead. The screw 870 may have a length SCL of, for example, 25 mm to 100 mm and may have a stem diameter SDL of, for example, 5.5 mm as well as a cap diameter of 5.5 mm.

Referring now to FIG. 25, lag screw 980 is shown in greater detail. Lag screw 980 includes a partially threaded stem 843 including a relief portion 984 and a threaded portion 982. The threaded portion 982 includes cancellous threads. The screw 841 further includes a cap 845. The stem 843 may have a diameter DL of, for example, 5.5 mm and may have a length LL, of, for example, 50 mm to 100 mm.

Referring now to FIG. 26, polyaxial cancellous screw assembly 934 is shown in greater detail. The screw assembly 934 includes screw 970 and bushing 924. The screw 970 is similar to the cancellous screw 70 of FIG. 14. The bushing 924 is similar to the bushing 24 of the screw assembly 34. Screw 970 is smaller than the screw 70 of FIG. 14.

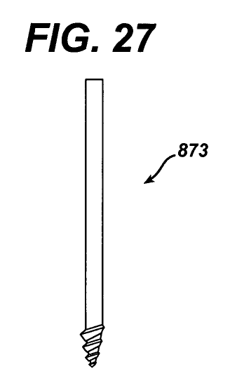

Referring now to FIG. 27, a k-wire 873 is shown. The k-wire 873 is suitable for use with the k-wire holes 871 and 971 of the plates 814 and 914, respectively. The k-wire has a generally cylindrical shaped body 893 with a cutting tip 895 located on an end thereof.

Referring now to FIG. 28, another embodiment of the present invention is shown as fracture repair system 1110. Fracture repair system 1110 is utilized for engagement with a bone, for example a femur 12 having a condylar portion 46 and a shaft portion 47 (see FIG. 19). The system 1110 includes a plate, for example, plate 814 including a head portion 844 and a body portion 842. The body portion 842 has an internal wall 846 defining a body hole 848 through the plate 814. The fracture repair system 1110 further includes a first rigid body attachment component 821, including a stem portion 823 for clearance passage through the body hole 848 and into the bone 12. The first rigid body attachment component, for example, rigid cortical screw 821 (see FIG. 23) further includes opposed cap portion 825 adapted to rigidly cooperate with the plate 814. The fracture repair system 1110 further includes a second rigid body attachment component 870 (see FIG. 24) including stem portion 870 for threadably engagement with the body hole 875 and into the bone 12 and an opposed cap portion 875 adapted to rigidly cooperate with the plate 814.

Fracture repair system 1110 provides for the use of the second fastener 870 when the first fastener 821 is stripped. Therefore, the stem 873 has a stem diameter SDL which is preferably larger than the stem diameter SDS of the stem 823 and in fact the stem 853 may be made of a coarse thread or a cancellous thread while the stem 873 may be a fine or cortical type thread.

Referring again to FIGS. 21 and 22, the fracture repair system of the present invention may alternatively utilize pins to replace at least a portion of the threaded fasteners of the repair system. Preferably the pins are secured to the plates 814 and 914 of FIGS. 21 and 22, respectively. Thus the pins are preferably used to replace the threaded fasters that have tapered threads that engage and lock to the plates 814 and 914. The pin may, for example have dimensions that are the same as the respective screw they replace including the same length and head configuration. The pins may have tapered threads adjacent the heads for securing the pin to the plate that are the same as the tapered threads of the respective screw. The pins may have a periphery on the pin shank that does not contain threads.

The diameter of the pin shank may be any diameter sufficient for proper strength. For example, the diameter of the pin shank may be the same as the respective screw thread major diameter or the minor diameter or, for example, any size in between. If a pin is used with the same diameter as the minor diameter of the respective screw the same drill may be used to prepare the pin as is used to prepare the hole for the screw. Also, a pin with a diameter equal to the minor diameter of the screw would have about the same strength as the screw, but be less invasive to the bone around where the pin is inserted than the respective screw.

For example and as shown in FIG. 21, the pin 821 A may be used to replace the screw 821 and the pin 70A may be used to replace the screw 70. Further the pin 870A may be used to replace the screw 870 and the pin 62A may be used to replace the screw 62.

Further, as shown in FIG. 22, the pin 934A may be used to replace the screw 934.

The pins may be installed by first preparing an opening in the bone for receiving the pin. A drill (not shown) may prepare the opening and a bushing (not shown) may be positioned over the hole in the plate to guide the drill. The drill may have the same diameter as the pin. The pin may be pushed into the drilled opening by any suitable method.

The pins may provide support for the plate in the longitudinal axis of the bone, transverse to the longitudinal axis of the pin. The pins may be easier to install than screws and may be less disruptive to the bone adjacent where they are installed.

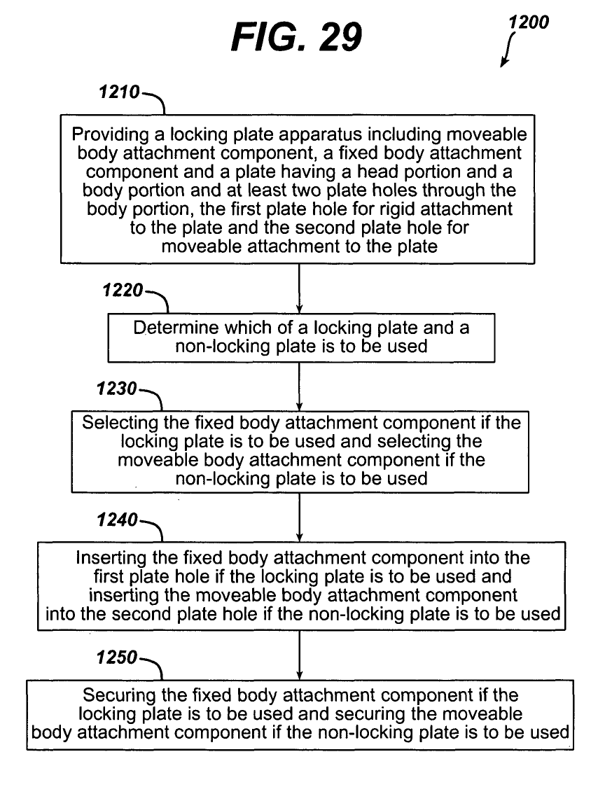

Referring now to FIG. 29, another embodiment of the present invention is shown as method 1200. The method 1200 is utilized for repairing a bone fracture on a bone having a condylar portion and a shaft portion. The method 1200 includes a first step 1210 of providing a locking plate apparatus including movable body attachment component, a fixed body attachment component and a plate. The plate includes a head portion and a body portion and at least two plate holes through the body portion, the first plate hole for rigid attachment to the plate and the second plate hole for movable attachment to the plate. The method further includes a second step 1220 of determining which of a locking or non-locking plate bone is to be used. The method 1200 also includes a third step 1230 of selecting the fixed body attachment component if a locking plate is to be used and selecting the movable body attachment component if a non-locking plate is to be used. The method 1200 further includes a fourth step 1240 of inserting the fixed body attachment component into the first plate hole if the locking plate is to be used and inserting the movable body attachment component into the second plate hole if the non-locking plate is to be used. The method includes a fifth step 1250 of securing the fixed body attachment component if the locking plate is to be used and securing the movable body attachment component if the non-locking plate is to be used.

By providing a fracture repair system including a bushing to permit polyaxial rotation of the bushing within the hole plate an attachment component may be secured to a plate with the ability to position divergently to secure the fracture of the bone most efficiently. For example bone fragments may be reached by orienting the attachment component relative to the plate in such a direction to reach various bone fragments.

By providing a fracture repair system including a bushing with a spherical outside diameter in cooperation with a plate having a spherical bore, a low-friction polyaxial rotation of the attachment component relative to the plate is possible.

By providing a fracture repair system including a bushing having a tapered threaded bore in cooperation with a tapered threaded or non-threaded attachment component, the attachment component may be rigidly secured in a variety of orientations.

By providing a fracture repair system including a polyaxial bushing which may be rigidly secured to a plate and including a closely conforming plate which closely conforms to the condyle areas of a long bone the fragments fractured components within the condyle areas may be effectively and efficiently contained.

By providing a fracture repair system including a threaded alignment hole for securing a jig for drilling and threading the plate to the bone perpendicularly, a simple to use effective efficient bone plate system can be provided.

By providing a bone plate including a contoured tip for percutaneous insertion, a bone plate may be provided percutaneously for minimally invasive surgery. Such a contoured tip permits easy and effective insertion and alignment of the plate to the bone.

Although the invention has been described in detail with reference to a preferred embodiment, variations and modifications exist within the scope and spirit of the invention as described and defined in the following claims.

What is claimed is:

1. A fracture repair system for engagement with a bone having a condylar portion and a shaft portion, the system comprising:
a plate including a head portion and a body portion, the head portion having an internal wall defining a head hole therethrough and adapted for cooperation with the condylar portion, the body portion having an internal wall defining a body hole therethrough;
a bushing including a generally spherical exterior surface adapted for cooperation with the head hole and an opposed interior surface defining a passageway therethrough, the exterior surface of said bushing and the head hole of said plate being configured to permit polyaxial rotation of said bushing within the head hole;
a head attachment component including a distal portion sized for clearance passage through the passageway and into the bone and an opposed proximate portion sized to urge said bushing against the internal wall of said plate to form a friction lock between said bushing and said plate in a selected polyaxial position, said head attachment component being positionable in an orientation extending divergingly from said plate; and
a body attachment component including a stem portion for passage through the body hole and into the bone and an opposed cap portion sized to cooperate with said plate.

2. A fracture repair system as in claim 1, wherein said plate defines a surface thereof, the surface closely conforming to the bone.

3. A fracture repair system as in claim 1:
wherein the body portion of said plate further defines a second body hole through the body portion; and
further comprising a second body attachment component including a stem portion for passage through the second body hole and into the bone and an opposed cap portion sized to cooperate with said plate.

4. A fracture repair system as in claim 3:
wherein the cap portion of said first mentioned body attachment component is fixedly securable to said plate; and
wherein the cap portion of said second body attachment component is moveably securable to said plate.

5. A fracture repair system as in claim 4, wherein the body portion of said plate adjacent the first mentioned body hole defines a first location featured for cooperating with a drill jig for guiding the attachment components and the body portion of said plate adjacent the second body hole defines a second location featured for cooperating with the drill jig, the first location feature and the second location feature being substantially identical.

6. A fracture repair system as in claim 1:
wherein said attachment component comprises first external threads on the proximate portion thereof and second external threads on the distal portion thereof, and
wherein the radially interior surface of said bushing comprises first internal threads thereon, said first internal threads of said bushing engageable with said first internal threads of said attachment component.

7. A fracture repair system for engagement with a bone having a condylar portion and a shaft portion, the system comprising:
a plate including a head portion and a body portion, the body portion having an internal wall defining a first body hole and a spaced apart second body hole therethrough;
a rigid body attachment component including a stem portion for passage through the first body hole and into the bone and an opposed cap portion adapted to rigidly cooperate with said plate; and
a movable body attachment component including a stem portion for passage through the second body hole and into the bone and an opposed cap portion adapted to movably cooperate with said plate.

8. A fracture repair system as in claim 7, wherein said plate defines a surface thereof, the surface closely conforming to the bone.

9. A fracture repair system as in claim 7:
wherein the stem portion of said first mentioned rigid body attachment component is sized to be in clearance with the body hole; and
further comprising a second rigid body attachment component including a stem portion for passage through the body hole and into the bone and an opposed cap portion adapted to rigidly cooperate with said plate, the stem portion of said second rigid body attachment component being threadably cooperable with said plate.

10. A fracture repair system as in claim 7:
wherein the head portion of said plate has an internal wall defining a head hole therethrough and adapted for cooperation with the condylar portion;
further comprising a bushing including a generally spherical exterior surface adapted for cooperation with the head hole and an opposed interior surface defining a passageway therethrough, the exterior surface of said bushing and the head hole of said plate being configured to permit polyaxial rotation of said bushing within the head hole; and
further comprising a head attachment component including a distal portion sized for clearance passage through the passageway and into the bone and an opposed proximate portion sized to urge said bushing against the internal wall of said plate to form a friction lock between said bushing and said plate in a selected polyaxial position, said head attachment component being positionable in an orientation extending divergingly from said plate.

11. A fracture repair system as in claim 7, wherein the body portion of said plate adjacent the first mentioned body hole defines a first location featured for cooperating with a drill jig for guiding the attachment components and the body portion of said plate adjacent the second body hole defines a second location featured for cooperating with the drill jig, the first location feature and the second location feature being substantially identical.

12. A fracture repair system for engagement with a bone having a condylar portion and a shaft portion, the system comprising:
a plate including a head portion and a body portion, the body portion having an internal wall defining a body hole therethrough;
a first rigid body attachment component including a stem portion for clearance passage through the body hole and into the bone and an opposed cap portion adapted to rigidly cooperate with said plate; and
a second rigid body attachment component including a stem portion for threadable engagment with the body hole and into the bone and an opposed cap portion adapted to rigidly cooperate with said plate.

13. A fracture repair system as in claim 12:
wherein the body portion of said plate further defines a second body hole therethrough; and
further comprising a movable body attachment component including a stem portion for passage through the second body hole and into the bone and an opposed cap portion adapted to movably cooperate with said plate.

14. A fracture repair system as in claim 13, wherein the body portion of said plate adjacent the first mentioned body hole defines a first location featured for cooperating with a drill jig for guiding the attachment components and the body portion of said plate adjacent the second body hole defines a second location featured for cooperating with the drill jig, the first location feature and the second location feature being substantially identical.

15. A fracture repair system as in claim 12, wherein said plate defines a surface thereof, the surface closely conforming to the bone.

16. A fracture repair system as in claim 12:
wherein the head portion of said plate has an internal wall defining a head hole therethrough and adapted for cooperation with the condylar portion;
further comprising a bushing including a generally spherical exterior surface adapted for cooperation with the head hole and an opposed interior surface defining a passageway therethrough, the exterior surface of said bushing and the head hole of said plate being configured to permit polyaxial rotation of said bushing within the head hole; and
further comprising a head attachment component including a distal portion sized for clearance passage through the passageway and into the bone and an opposed proximate portion sized to urge said bushing against the internal wall of said plate to form a friction lock between said bushing and said plate in a selected polyaxial position, said head attachment component being positionable in an orientation extending divergingly from said plate.

17. A fracture repair system for engagement with a bone, the system comprising:
a plate including a portion having an internal wall defining a first body hole and a spaced apart second body hole therethrough;
a rigid body attachment component including a stem portion for passage through the body hole and into the bone and an opposed cap portion adapted to rigidly cooperate with said plate; and
a movable body attachment component including a stem portion for passage through the body hole and into the bone and an opposed cap portion adapted to movably cooperate with said plate.

18. A fracture repair system as in claim 17, wherein said plate defines a surface thereof, the surface closely conforming to the bone.

19. A fracture repair system as in claim 17:
wherein the stem portion of said first mentioned rigid body attachment component is sized to be in clearance with the body hole; and
further comprising a second rigid body attachment component including a stem portion for passage through the body hole and into the bone and an opposed cap portion adapted to rigidly cooperate with said plate, the stem portion of said second rigid body attachment component threadably cooperates with said plate.

20. A fracture repair system as in claim 17:
wherein said plate defines a third hole therethrough;
further comprising a bushing including a generally spherical exterior surface adapted for cooperation with the third hole and an opposed interior surface defining a passageway therethrough, the exterior surface of said bushing and the third hole of said plate being configured to permit polyaxial rotation of said bushing within the third hole; and
further comprising a head attachment component including a distal portion sized for clearance passage through the passageway and into the bone and an opposed proximate portion sized to urge said bushing against the internal wall of said plate to form a friction lock between said bushing and said plate in a selected polyaxial position, said head attachment component being positionable in an orientation extending divergingly from said plate.

21. A fracture repair system as in claim 17, wherein said plate adjacent the first body hole defines a first location featured for cooperating with a drill jig for guiding the attachment components and said plate adjacent the second body hole defines a second location feature for cooperating with the drill jig, the first location feature and the second location feature being substantially identical.

22. A joint fracture system for use with joint having adjoining first and second long bones, said system comprising:
a first plate for cooperation with the first long bone, the first plate including a first plate head portion and a first plate body portion, the first plate body portion having an internal wall defining a first plate first body hole and a spaced apart first plate second body hole therethrough;
a first plate rigid body attachment component including a stem portion for passage through the first plate first body hole and into the bone and an opposed cap portion adapted to rigidly cooperate with said first plate;
a first plate movable body attachment component including a stem portion for passage through the first plate first body hole and into the bone and an opposed cap portion adapted to movably cooperate with said first plate;
a second plate for cooperation with the second long bone, the second plate including a second plate head portion and a second plate body portion, the second plate body portion having an internal wall defining a second plate first body hole and a spaced apart second plate second body hole therethrough;
a second plate rigid body attachment component including a stem portion for passage through the second plate first body hole and into the bone and an opposed cap portion adapted to rigidly cooperate with said second plate; and
a second plate movable body attachment component including a stem portion for passage through the second plate second body hole and into the bone and an opposed cap portion adapted to movably cooperate with said second plate.

23. A joint fracture repair system as in claim 22, wherein said plate defines a surface thereof, the surface closely conforming to the bone.

24. A joint fracture repair system as in claim 22:
wherein a portion of at least one of the stem portion of said rigid body attachment components for passage through the body hole is sized to be in clearance with the body hole; and
further comprising an additional rigid body attachment component including a stem portion for passage through the body hole and into the bone and an opposed cap portion adapted to rigidly cooperate with said plate, the stem portion of said second rigid body attachment component threadably cooperates with said plate.

25. A fracture repair system as in claim 22:
wherein the head portion of at least one of said plates has an internal wall defining a head hole therethrough and adapted for cooperation with the condylar portion;
further comprising a bushing including a generally spherical exterior surface adapted for cooperation with the head hole and an opposed interior surface defining a passageway therethrough, the exterior surface of said bushing and the head hole of said plate being configured to permit polyaxial rotation of said bushing within the head hole; and
further comprising a head attachment component including a distal portion sized for clearance passage through the passageway and into the bone and an opposed proximate portion sized to urge said bushing against the internal wall of said plate to form a friction lock between said bushing and at least one of said plates in a selected polyaxial position, said head attachment component being positionable in an orientation extending divergingly from at least one of said plates.

26. A fracture repair system as in claim 22, wherein at least one the body portion of said first plate and the body portion of said second plate defines a plate hole therethough opposed to the head portion, the plate hole adapted for cooperation with one of said first plate rigid body attachment component and said second plate rigid plate attachment to provide rigid attachment of said plate to said component to avoid movement of said plate with respect to the bone as the joint is moved.

27. A fracture repair system as in claim 22, wherein at least one the body portion of said first plate and the body portion of said second plate defines a end thereof opposed to the head portion, the end having a tapered shape to assist in percutaneous insertion of the plate into an implanting position adjacent one of the long bones.

28. A method for repairing a bone fracture on a bone having a condylar portion and a shaft portion, the method including the steps of:
providing a locking plate apparatus including moveable body attachment component, a fixed body attachment component and a plate having a head portion and a body portion and at least two plate holes through the body portion, the first plate hole for rigid attachment to the plate and the second plate hole for moveable attachment to the plate;
determining which of a locking plate and a non-locking plate bone is to be used;
selecting the fixed body attachment component if the locking plate is to be used and selecting the moveable body attachment component if the non-locking plate is to be used;
inserting the fixed body attachment component into the first plate hole if the locking plate is to be used and inserting the moveable body attachment component into the second plate hole if the non-locking plate is to be used; and
securing the fixed body attachment component if the locking plate is to be used and securing the moveable body attachment component if the non-locking plate is to be used.

29. The method of claim 28, wherein the step of determining which of a locking plate and a non-locking plate is used is based on determining which of an osteoporotic bone and a non-osteoporotic bone is to be repaired, the locking plate to be used if the bone is osteoporotic and the non-locking plate to be used if the bone is non-osteoporotic.

30. A fracture repair system for engagement with a bone having a condylar portion and a shaft portion, the system comprising:
a plate including a head portion and a body portion, the head portion having an internal wall defining a head hole therethrough and adapted for cooperation with the condylar portion, the body portion having an internal wall defining a body hole therethrough; and
an attachment component including a distal portion sized for passage through at least one of the head hole and the body hole and into the bone and an opposed proximate portion sized to rigidly secure to the internal wall of one of the head hole and the body hole, the distal portion being generally cylindrical and having a smooth periphery.

31. A fracture repair system as in claim 30, wherein said plate defines a surface thereof, the surface closely conforming to the bone.

32. A fracture repair system as in claim 30, further comprising a second attachment component including a distal portion sized for passage through the other of the head hole and the body hole and into the bone and an opposed proximate portion sized to rigidly secure to the internal wall of the other of the head hole and the body hole, the distal portion being generally cylindrical and having a smooth periphery.

33. A fracture repair system as in claim 30, further comprising a second attachment component having first external threads on the proximate portion thereof and second external threads on the distal portion thereof.
